(19) United States
(12) Patent Application Publication
(10) Pub. No.: US 2025/0318420 A1
(43) Pub. Date: Oct. 9, 2025

(54) PSEUDO-HOMOGENEOUS PHOTO-PATTERNABLE SEMICONDUCTING POLYMER BLENDS FOR ORGANIC THIN-FILM TRANSISTORS (OTFT)

(71) Applicants: CORNING INCORPORATED, CORNING, NY (US); FUDAN UNIVERSITY, Shanghai (CN)

(72) Inventors: Renzhong Chen, Wenzhou City (CN); Mingqian He, Horseheads, NY (US); Xin Li, Shanghai (CN); Yang Li, Painted Post, NY (US); Dacheng Wei, Shanghai (CN)

(21) Appl. No.: 18/867,901

(22) PCT Filed: May 23, 2023
(86) PCT No.: PCT/US2023/023163

§ 371 Date: Nov. 21, 2024

(30) Foreign Application Priority Data
May 30, 2022 (CN) 202210601414.4

(51) Int. Cl. H10K 85/10; G03F 7/00; G03F 7/038; G03F 7/039; G03F 7/16; G03F 7/20; H10K 10/46

(52) U.S. Cl. CPC H10K 85/113; G03F 7/0382; G03F 7/0392; G03F 7/168; G03F 7/2002; G03F 7/70008; H10K 85/151; H10K 10/46

(57) ABSTRACT

A semiconductor device, including at least one organic semiconductor (OSC) polymer and at least one photosensitizer, such that the at least one OSC polymer is a diketopyrrolopyrrole-fused thiophene polymeric material, and the fused thiophene is beta-substituted.

### CROSS-REFERENCE TO RELATED APPLICATIONS

This application claims the benefit of priority to Chinese Patent Application 202210601414.4 filed on May 30, 2022, which is incorporated herein by reference in its entirety for all purposes.

### BACKGROUND

### 1. Field

This disclosure relates to pseudo-homogeneous photo-patternable semiconducting polymer blends for organic thin-film transistors (OTFTs).

### 2. Technical Background

Organic thin-film transistors (OTFTs) have garnered extensive attention as alternatives to conventional silicon-based technologies, which require high temperature and high vacuum deposition processes, as well as complex photolithographic patterning methods. Semiconducting (i.e., organic semiconductor, OSC) layers are one important component of OTFTs which can effectively influence the performance of devices.

Traditional technologies in the manufacture of inorganic TFT device arrays often rely on photolithography as the patterning process. However, photolithography usually involves harsh oxygen (O₂) plasma during pattern transfer or photoresist removal and aggressive developing solvents which may severely damage the OSC layer and lead to significant deterioration of device performance.

This disclosure presents improved pseudo-homogeneous photo-patternable semiconducting polymer blends and use thereof for OSC layers of organic thin-film transistors.

### SUMMARY

In embodiments, a semiconductor device, comprises at least one organic semiconductor (OSC) polymer and at least one photosensitizer, wherein the at least one OSC polymer is a diketopyrrolopyrrole-fused thiophene polymeric material, wherein the fused thiophene is beta-substituted.

In aspects, which are combinable with any of the other aspects or embodiments, the at least one OSC polymer comprises a first OSC polymer and a second OSC polymer. In aspects, which are combinable with any of the other aspects or embodiments, the first OSC polymer and the second OSC polymer have identical conjugated backbones. In aspects, which are combinable with any of the other aspects or embodiments, a weight ratio between the first OSC polymer and the second OSC polymer ranges from 4:1 to 1:4. In aspects, which are combinable with any of the other aspects or embodiments, the semiconductor device comprises an isotropic charge mobility of at least 0.40 cm² V⁻¹ s⁻¹. In aspects, which are combinable with any of the other aspects or embodiments, the semiconductor device comprises a bottom-gate bottom-contact (BGBC)-configurated organic thin film transistor (OTFT) array.

### BRIEF DESCRIPTION OF THE DRAWINGS

The disclosure will become more fully understood from the following detailed description, taken in conjunction with the accompanying figures, in which:

FIG. 1 illustrates polymer structures of semiconducting polymers PTDPPTFT4-zC, x and y are relative mole ratio of DPP monomers, according to embodiments.

FIG. 2 illustrates polymer structures of methacrylate functionalized semiconducting polymer X-190401 and alkyl side chain semiconducting polymer C255, according to embodiments.

FIG. 3 illustrates contrast curves of SP-2 and PTDPPTFT4-5C, according to embodiments.

FIGS. 4A-4C illustrate optical microscopy (OM) images of line-like patterns of SP-2 and FIGS. 4D-4F illustrate OM images of line-like patterns of PTDPPTFT4-5C, according to embodiments. Scale bar is 5 μm.

FIG. 5A illustrates atomic force microscopy (AFM) images of square-like patterns of SP-2 and FIG. 5B illustrates AFM images of square-like patterns of PTDPPTFT4-5C, according to embodiments. Scale bar is 2.5 μm.

FIG. 6 illustrates the influence of cinnamate-DPP ratios on charge mobilities of SP-2 and PTDPPTFT4-zC, according to embodiments.

FIG. 7 illustrates processing and environmental stabilities of SP-2/50 and PTDPPTFT4-5C, according to embodiments.

FIG. 8 illustrates five (5) concentric circular OTFT arrays with increasing channel widths of 1, 5, 10, 20, and 50 μm, respectively, according to embodiments. Scale bar is 5 mm.

FIG. 9A illustrates charge mobilities and FIG. 9B illustrates an OM image of SP-2/50 at various channel widths with channel direction perpendicular to the centrifugal force, according to embodiments. Scale bar is 500 μm.

FIG. 10A illustrates charge mobilities and FIG. 10B illustrates an OM image of SP-1 at various channel widths with channel direction perpendicular to the centrifugal force, according to embodiments. Scale bar is 500 μm.

FIG. 11A illustrates charge mobilities of SP-2/50 at various channel widths with channel direction perpendicular to the centrifugal force and FIG. 11B illustrates charge mobilities of SP-2/50 at various channel widths with channel direction parallel to the centrifugal force, according to embodiments.

FIG. 12 illustrates an OM image of a photo patterned SP-2/50 line in a bottom-gate bottom-contact (BGBC) OTFT of 0.9 μm width according to embodiments. Scale bar is 20 μm.

FIGS. 13A and 13B illustrate transmission electron microscopy (TEM) images of PTDPPTFT4-5C (FIG. 13A) and SP-2/50 (FIG. 13B), according to embodiments. Scale bar is 100 nm.

FIGS. 14A and 14B illustrate schematic illustrations of molecule aggregates of PTDPPTFT4-5C (FIG. 14A) and SP-2 (FIG. 14B), according to embodiments.

FIG. 15 illustrates an optical microscopy (OM) image of a BGBC-configurated OTFT array with high device density up to 106 units/cm², according to embodiments. The photo patterned SP-2/50 has line width as small as 0.8 μm. Scale bar is 20 μm.

FIGS. 16A-16F illustrate OM images of down-scaled OTFTs (FIGS. 16A, 16B), PMOS inverters (FIGS. 16C, 16D), and 3-stage ring oscillators (FIGS. 16E, 16F), according to embodiments. FIGS. 16A, 16C, and 16E are original devices, while FIGS. 16B, 16D, and 16F are shrunken devices.

FIGS. 17A-17C illustrate electrical performance of down-scaled OTFTs (FIG. 17A), PMOS inverters (FIG. 17B), and 3-stage ring oscillators (FIG. 17C), according to embodiments. The red line are original devices and the green line are shrunken devices.

FIG. 18 illustrates an OM image of UV patterns of C255/X-190401 blend (fresh solution) (left) and an OM image of UV patterns of C255/X-190401 blend (7-day aged solution at room temperature) (right), according to embodiments.

FIGS. 19A-19E illustrate traditional patterning techniques of organic semiconductor materials utilizing photoresists.

FIGS. 20A-20C illustrate patterning techniques of organic semiconductor materials, according to embodiments.

FIG. 21 illustrates an exemplary OTFT device, according to embodiments.

FIG. 22 illustrates an exemplary OTFT device, according to embodiments.

### DETAILED DESCRIPTION

Reference will now be made in detail to exemplary embodiments which are illustrated in the accompanying drawings. Whenever possible, the same reference numerals will be used throughout the drawings to refer to the same or like parts. The components in the drawings are not necessarily to scale, emphasis instead being placed upon illustrating the principles of the exemplary embodiments. It should be understood that the present application is not limited to the details or methodology set forth in the description or illustrated in the figures. It should also be understood that the terminology is for the purpose of description only and should not be regarded as limiting.

Additionally, any examples set forth in this specification are illustrative, but not limiting, and merely set forth some of the many possible embodiments of the claimed invention. Other suitable modifications and adaptations of the variety of conditions and parameters normally encountered in the field, and which would be apparent to those skilled in the art, are within the spirit and scope of the disclosure.

### Definitions

The term ‘cinnamate’ refers to a salt or ester of cinnamic acid, which is an organic compound with the formulate C₆H₅CH═CHCOOH. Both cinnamic acids and cinnamates are classified as unsaturated carboxylic acids. Cinnamates may occur as both cis and trans isomers.

The term ‘chalcone’ refers to an aromatic ketone and an enone that forms the central core for a variety of important biological compounds, collectively as chalcones or chalconoids. Examples of chalcones include benzylideneacetophenone, phenyl styryl ketone, benzalacetophenone, β-phenylacrylophenone, γ-oxo-α,γ-diphenyl-α-propylene, and α-phenyl-β-benzoylethylene.

The term ‘coumarin’ (i.e., 2H-chromen-2-one) refers to an aromatic organic chemical compound with formula C9H₆O₂. It is a benzene molecule with two adjacent hydrogen atoms replaced by a lactone-like chain —O—, forming a second six-membered heterocycle that shares two carbons with the benzene ring. It may be placed in the benzopyrone chemical class and considered as a lactone.

The term ‘arylalkene’ refers to an alkene group that is directly bonded to an aromatic group.

The term “alkyl group” refers to a monoradical branched or unbranched saturated hydrocarbon chain having 1 to 40 carbon atoms. This term is exemplified by groups such as methyl, ethyl, n-propyl, isopropyl, n-butyl, isobutyl, t-butyl, pentyl, n-hexyl, n-heptyl, n-octyl, n-decyl, or tetradecyl, and the like. The alkyl group can be substituted or unsubstituted.

The term “substituted alkyl group” refers to: (1) an alkyl group as defined above, having 1, 2, 3, 4 or 5 substituents, typically 1 to 3 substituents, selected from the group consisting of alkenyl, alkynyl, alkoxy, aralkyl, aldehyde, cycloalkyl, cycloalkenyl, acyl, acylamino, acyl halide, acyloxy, amino, aminocarbonyl, alkoxycarbonylamino, azido, cyano, halogen, hydroxy, keto, thiocarbonyl, carboxy, carboxyalkyl, arylthiol, ester, heteroarylthio, heterocyclylthio, hydroxyl, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl, thioalkyl, vinyl ether. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2; or (2) an alkyl group as defined above that is interrupted by 1-10 atoms independently chosen from oxygen, sulfur and NRₐ, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR_{SO}, in which R_{SO} is alkyl, aryl, or heteroaryl and n is 0, 1 or 2; or (3) an alkyl group as defined above that has both 1, 2, 3, 4 or 5 substituents as defined above and is also interrupted by 1-10 atoms as defined above. For example, the alkyl groups can be an alkyl hydroxy group, where any of the hydrogen atoms of the alkyl group are substituted with a hydroxyl group.

The term “alkyl group” as defined herein also includes cycloalkyl groups. The term “cycloalkyl group” as used herein is a non-aromatic carbon-based ring (i.e., carbocyclic) composed of at least three carbon atoms, and in some embodiments from three to 20 carbon atoms, having a single cyclic ring or multiple condensed rings. Examples of single ring cycloalkyl groups include, but are not limited to, cyclopropyl, cyclobutyl, cyclopentyl, cyclohexyl, cyclooctyl, and the like. Examples of multiple ring cycloalkyl groups include, but are not limited to, adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like. The term cycloalkyl group also includes a heterocycloalkyl group, where at least one of the carbon atoms of the ring is substituted with a heteroatom such as, but not limited to, nitrogen, oxygen, sulfur, or phosphorus.

The term “unsubstituted alkyl group” is defined herein as an alkyl group composed of just carbon and hydrogen.

The term “acyl” denotes a group —C(O)R_{CO}, in which R_{CO} is hydrogen, optionally substituted alkyl, optionally substituted cycloalkyl, optionally substituted heterocyclyl, optionally substituted aryl, and optionally substituted heteroaryl.

The term “aryl group” as used herein is any carbon-based aromatic group (i.e., aromatic carbocyclic) such as having a single ring (e.g., phenyl) or multiple rings (e.g., biphenyl), or multiple condensed (fused) rings (e.g., naphthyl or anthryl). These may include, but are not limited to, benzene, naphthalene, phenyl, etc.

The term “aryl group” also includes “heteroaryl group,” meaning a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen, sulfur, and phosphorus within at least one ring. In other words, heteroaryl groups are aromatic rings composed of at least three carbon atoms that has at least one heteroatom incorporated within the ring of the aromatic group. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, triazole, oxazole, thiazole, naphthyridine, and the like as well as N-oxide and N-alkoxy derivatives of nitrogen containing heteroaryl compounds, for example pyridine-N-oxide derivatives.

Unless otherwise constrained by the definition for the heteroaryl substituent, such heteroaryl groups can be optionally substituted with 1 to 5 substituents, typically 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The aryl group can be substituted or unsubstituted. Unless otherwise constrained by the definition for the aryl substituent, such aryl groups can optionally be substituted with from 1 to 5 substituents, typically 1 to 3 substituents, selected from the group consisting of alkyl, alkenyl, alkynyl, alkoxy, aldehyde, cycloalkyl, cycloalkenyl, acyl, acylamino, acyloxy, amino, aminocarbonyl, alkoxycarbonylamino, azido, cyano, ester, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2. In some embodiments, the term “aryl group” is limited to substituted or unsubstituted aryl and heteroaryl rings having from three to 30 carbon atoms.

The term “aralkyl group” as used herein is an aryl group having an alkyl group or an alkylene group as defined herein covalently attached to the aryl group. An example of an aralkyl group is a benzyl group. “Optionally substituted aralkyl” refers to an optionally substituted aryl group covalently linked to an optionally substituted alkyl group or alkylene group. Such aralkyl groups are exemplified by benzyl, phenylethyl, 3-(4-methoxyphenyl) propyl, and the like.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “alkenyl group” refers to a monoradical of a branched or unbranched unsaturated hydrocarbon group typically having from 2 to 40 carbon atoms, more typically 2 to 10 carbon atoms and even more typically 2 to 6 carbon atoms and having 1-6, typically 1, double bond (vinyl). Typical alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. When alkenyl is attached to nitrogen, the double bond cannot be alpha to the nitrogen.

The term “substituted alkenyl group” refers to an alkenyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “cycloalkenyl group” refers to carbocyclic groups of from 3 to 20 carbon atoms having a single cyclic ring or multiple condensed rings with at least one double bond in the ring structure.

The term “alkynyl group” refers to a monoradical of an unsaturated hydrocarbon, typically having from 2 to 40 carbon atoms, more typically 2 to 10 carbon atoms and even more typically 2 to 6 carbon atoms and having at least 1 and typically from 1-6 sites of acetylene (triple bond) unsaturation. Typical alkynyl groups include ethynyl, (—C≡CH), propargyl (or prop-1-yn-3-yl, —CH₂C≡CH), and the like. When alkynyl is attached to nitrogen, the triple bond cannot be alpha to the nitrogen.

The term “substituted alkynyl group” refers to an alkynyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “alkylene group” is defined as a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, typically 1-10 carbon atoms, more typically 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene group” refers to: (1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2; or (2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocyclyl, or groups selected from carbonyl, carboxyester, carboxyamide and sulfonyl; or (3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), and the like.

The term “alkoxy group” refers to the group R—O—, where R is an optionally substituted alkyl or optionally substituted cycloalkyl, or R is a group —Y—Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio group” refers to the group R_{S}—S—, where R_{S} is as defined for alkoxy.

The term “aminocarbonyl” refers to the group —C(O)NR_{N}R_{N} where each R_{N} is independently hydrogen, alkyl, aryl, heteroaryl, heterocyclyl or where both R_{N} groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “acylamino” refers to the group —NR_{NCO}C(O)R where each R_{NCO} is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “aryloxy group” refers to the group aryl-O— wherein the aryl group is as defined above, and includes optionally substituted aryl groups as also defined above.

The term “heteroaryloxy” refers to the group heteroaryl-O—.

The term “amino” refers to the group —NH₂.

The term “substituted amino” refers to the group —NR_{W}R_{W} where each R_{W} is independently selected from the group consisting of hydrogen, alkyl, cycloalkyl, carboxyalkyl (for example, benzyloxycarbonyl), aryl, heteroaryl and heterocyclyl provided that both R_{W} groups are not hydrogen, or a group —Y—Z, in which Y is optionally substituted alkylene and Z is alkenyl, cycloalkenyl, or alkynyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “carboxy” refers to a group —C(O)OH. The term “carboxyalkyl group” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, and —S(O)ₙR_{SO}, in which R_{SO} is alkyl, aryl, or heteroaryl and n is 0, 1 or 2.

The terms “substituted cycloalkyl group” or “substituted cycloalkenyl group” refer to cycloalkyl or cycloalkenyl groups having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “conjugated group” is defined as a linear, branched or cyclic group, or combination thereof, in which p-orbitals of the atoms within the group are connected via delocalization of electrons and wherein the structure can be described as containing alternating single and double or triple bonds and may further contain lone pairs, radicals, or carbenium ions. Conjugated cyclic groups may comprise both aromatic and non-aromatic groups, and may comprise polycyclic or heterocyclic groups, such as diketopyrrolopyrrole. Ideally, conjugated groups are bound in such a way as to continue the conjugation between the thiophene moieties they connect. In some embodiments, “conjugated groups” is limited to conjugated groups having three to 30 carbon atoms.

The term “halogen,” “halo,” or “halide” may be referred to interchangeably and refer to fluoro, bromo, chloro, and iodo.

The term “heterocyclyl” refers to a monoradical saturated or partially unsaturated group having a single ring or multiple condensed rings, having from 1 to 40 carbon atoms and from 1 to 10 hetero atoms, typically 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclyl substituent, such heterocyclyl groups can be optionally substituted with 1, 2, 3, 4 or 5, and typically 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “thiol” refers to the group —SH. The term “substituted alkylthio” refers to the group —S-substituted alkyl. The term “arylthiol group” refers to the group aryl-S—, where aryl is as defined as above. The term “heteroarylthiol” refers to the group —S-heteroaryl wherein the heteroaryl group is as defined above including optionally substituted heteroaryl groups as also defined above.

The term “sulfoxide” refers to a group —S(O)R_{SO}, in which R_{SO} is alkyl, aryl, or heteroaryl. The term “substituted sulfoxide” refers to a group —S(O)R_{SO}, in which R_{SO} is substituted alkyl, substituted aryl, or substituted heteroaryl, as defined herein. The term “sulfone” refers to a group —S(O)₂R_{SO}, in which R_{SO} is alkyl, aryl, or heteroaryl. The term “substituted sulfone” refers to a group —S(O)₂R_{SO}, in which R_{SO} is substituted alkyl, substituted aryl, or substituted heteroaryl, as defined herein.

The term “keto” refers to a group —C(O)—. The term “thiocarbonyl” refers to a group —C(S)—.

As used herein, the term “room temperature” is 20° C. to 25° C.

Disclosed are compounds, compositions, and components that can be used for, can be used in conjunction with, can be used in preparation of, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A weight percent of a component, unless specifically stated to the contrary, is based on the total weight of the formulation or composition in which the component is included.

Organic semiconductors as functional materials may be used in a variety of applications including, for example, printed electronics, organic transistors, including organic thin-film transistors (OTFTs) and organic field-effect transistors (OFETs), organic light-emitting diodes (OLEDs), organic integrated circuits, organic solar cells, and disposable sensors. Organic transistors may be used in many applications, including smart cards, security tags, and the backplanes of flat panel displays. Organic semiconductors may substantially reduce cost compared to inorganic counterparts, such as silicon. Depositing OSCs from solution may enable fast, large-area fabrication routes such as various printing methods and roll-to-roll processes.

Organic thin-film transistors are particularly interesting because their fabrication processes are less complex as compared with conventional silicon-based technologies. For example, OTFTs generally rely on low temperature deposition and solution processing, which, when used with semiconducting conjugated polymers, can achieve valuable technological attributes, such as compatibility with simple-write printing techniques, general low-cost manufacturing approaches, and flexible plastic substrates. Other potential applications for OTFTs include flexible electronic papers, sensors, memory devices (e.g., radio frequency identification cards (RFIDs)), remote controllable smart tags for supply chain management, large-area flexible displays, and smart cards.

For all organic devices and most integrated organic circuits, a patterned semiconducting layer generally provides two benefits over continuous layers. First, a patterned active layer in OTFTs reduces or eliminates parasitic current paths (cross-talk) between neighboring devices, leading to an improved on/off ratio, which is a significant merit impacting the contrast ratio in analog applications and noise level in digital applications. Patterning is even more important when devices share a common gate, through which the leakage current is significant. Second, a patterned semiconductor film removes material from the inactive areas for the optical path or deposition of subsequent functional layers.

Unfortunately, conventional photolithography for inorganic semiconductors is not applicable to most organic semiconductors because exposing to photoresists and developer/stripper cause inevitable damages to active layers and OTFTs, thereby significantly degrading their electrical and optical properties. Numerous efforts have been dedicated to developing novel patterning methods, in addition to photolithography, for organic semiconductors. Inkjet printing and nanoimprinting lithography are the most promising techniques; however, inkjet printing suffers from limited pattern resolutions and slow processing speed. The drawback is especially profound for highly integrated circuits on large area substrates. Nanoimprinting resolutions can reach as high as <100 nm; however, the process is multi-stepped and time-consuming. Furthermore, it is still a lab-scale process which lacks mature commercial equipment to enable low-cost and automatic mass production. In addition, for electronic devices fabricated by consecutive solution-deposition of organic layers, both inkjet printing and nanoimprinting methods suffer from poor chemical/solvent resistance of the patterned layers, resulting in deteriorated device performance, poor reliability and reproducibility.

With respect to semiconductor industry, high-throughput, reliable and well-established manufacturing methods are of critical importance for commercial success. Therefore, while a variety of patterning methods have been developed for organic semiconductors (as described above), efforts in material and process development to render organic semiconductors compatible with well-established photolithography has never stopped.

UV-patternable blends composed of UV crosslinkable acrylate crosslinker and a semiconducting polymer have been previously developed. This semiconducting photoresist (termed as SP-1) featured high patterning resolutions, superior chemical resistance, outstanding electrical performance, and high OTFT integration density up to 10⁵ units/cm². Examples of SP-1 include, without limitation, those described in U.S. Publication No. 2021/0341838A1, UV PATTERNABLE POLYMER BLENDS FOR ORGANIC THIN-FILM TRANSISTORS, which is assigned to Corning, Inc. and incorporated by reference herein in its entirety. However, as channel widths decreased to below 10 μm, SP-1's submicron phase separated structures (>150 nm) resulted in unsatisfactory device-to-device uniformity and dramatically reduced charge mobilities.

Prior to SP-1, other UV-patternable semiconducting polymers included conjugated backbone tetrathiophene-diketopyrrolopyrrole (FT4-DPP) having various UV-sensitive side chains engineered on the DPP moiety. Five functionalized DPP monomers were synthesized and incorporated into the FT4-DPP semiconducting polymers via conventional Still reaction. According to reaction mechanisms, the UV patternable FT4-DPP polymers were classified into three categories: radical crosslinking for acrylate/methyl acrylate DPP, [2+2] cycloaddition for coumarin/cinnamate DPP, and deprotection approach for t-Boc DPP. However, photo-crosslinkable semiconducting polymers based on side chain functionalization only had limited success in photolithographically patterned OTFTs because of their low patterning resolutions, unsatisfactory electrical performance, and poor environmental stability, In addition, though patterning of crystalline organic semiconductors may be achieved with high charge mobilities and sub-micron resolutions, poor device-to-device uniformity resulted from structural anisotropy hindered their industrial applications.

The miniaturization and integration of all-photolithography organic electronics require a reliable semiconducting photoresist with a high effective patterning resolution (EPR). EPR is defined as the critical dimension that clearly distinguishes two adjacent feature patterns while maintaining uncompromised and uniform electrical performance simultaneously. Semiconducting photoresists manufactured at or below EPR can produce precise patterns with predictable electrical characteristics, ensuring circuit design and manufacturing viability. No existing photo-crosslinkable organic semiconducting polymers or semiconducting photoresists can achieve EPR below 10 μm, which is a big hurdle for all-photolithography organic electronics to further reduce device dimensions and improve integration densities.

### Organic Semiconductor (OSC) Polymer

An OSC polymer may be used to produce organic semiconductor devices. In examples, a polymer blend comprises an organic semiconductor polymer. In examples, the OSC polymer has a main backbone that is fully conjugated. In examples, the OSC is a diketopyrrolopyrrole (DPP) fused thiophene polymeric material. In examples, the fused thiophene is beta-substituted. This OSC may contain both fused thiophene and diketopyrrolopyrrole units. In examples, the OSC is used in OTFT applications. For example, the OSC polymer may comprise the repeat unit of Formula 1 or Formula 2, or a salt, isomer, or analog thereof:

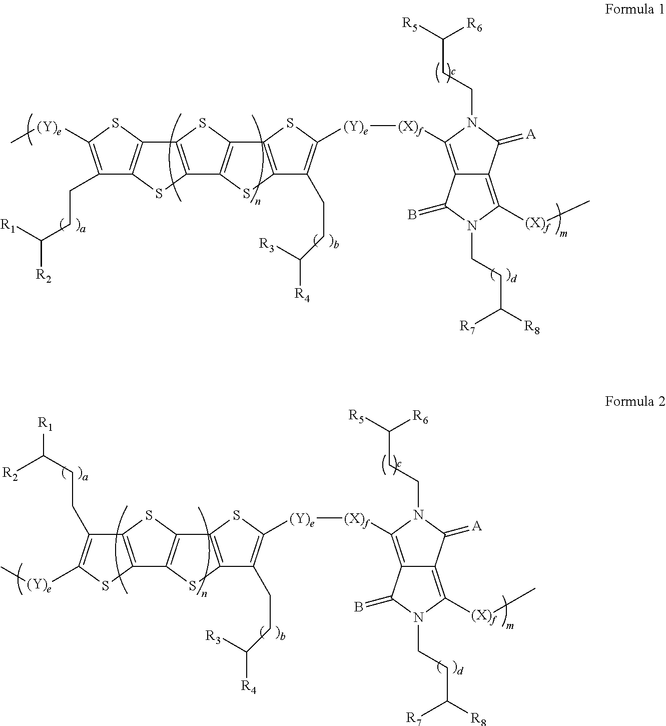

wherein in Formula 1 and Formula 2: m is an integer greater than or equal to one; n is 0, 1, or 2; R₁, R₂, R₃, R₄, R₅, R₆, R₇, and R₈, may be, independently, hydrogen, substituted or unsubstituted C₄ or greater alkyl, substituted or unsubstituted C₄ or greater alkenyl, substituted or unsubstituted C₄ or greater alkynyl, or C₅ or greater cycloalkyl; a, b, c, and d are independently, integers greater than or equal to 3; e and f independently are integers greater than or equal to zero; X and Y are, independently a covalent bond, an optionally substituted aryl group, an optionally substituted heteroaryl, an optionally substituted fused aryl or fused heteroaryl group, an alkyne or an alkene; and A and B may be, independently, either S or O, with the provisos that: (i) at least one of R₁ or R₂; one of R₃ or R₄; one of R₅ or R₆; and one of R₇ or R₈ is a substituted or unsubstituted alkyl, substituted or unsubstituted alkenyl, substituted or unsubstituted alkynyl, or cycloalkyl; (ii) if any of R₁, R₂, R₃, or R₄ is hydrogen, then none of R₅, R₆, R₇, or R₈ are hydrogen; (iii) if any of R₅, R₆, R₇, or R₈ is hydrogen, then none of R₁, R₂, R₃, or R₄ are hydrogen; (iv) e and f cannot both be 0; (v) if either e or f is 0, then c and d, independently, are integers greater than or equal to 5; and (iv) an OSC polymer of the at least one OSC polymer has a molecular weight, wherein the molecular weight of the OSC polymer is greater than 10,000.

In embodiments, the OSC polymers defined in Formula 1 or Formula 2 enable simple transistor fabrication at relatively low temperatures, which is particularly important for the realization of large-area, mechanically flexible electronics. A beta-substituted OSC polymer can also help to improve solubility.

In examples, an OSC polymer may comprise a first portion and a second portion, such that at least one of the first portion or the second portion comprises at least one UV-curable side chain. In examples, the at least one UV-curable side chain comprises at least one of acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or combinations thereof. In examples, only the first portion comprises the at least one UV-curable side chain. In examples, only the second portion comprises the at least one UV-curable side chain. In examples, both the first portion and the second portion comprise the at least one UV-curable side chain.

In examples, such as when the first portion comprises the at least one UV-curable side chain, the second portion comprises a repeat unit of Formulas 3-6, or a salt, isomer, or analog thereof. In examples, such as when the second portion comprises the at least one UV-curable side chain, the first portion comprises a repeat unit of Formulas 3-6, or a salt, isomer, or analog thereof. In examples, R₅ and R₇ are hydrogen and R₆ and R₈ are substituted or unsubstituted C₄ or greater alkenyl in the first portion and the second portion comprises a repeat unit of Formulas 3-6, or a salt, isomer, or analog thereof. In examples, each n in Formulas 3-5 independently is an integer greater than or equal to 1, and optionally less than or equal to 150 or less than or equal to 100. In examples, a and b in Formula 6 independently are an integer greater than or equal to 0 (provided that one of a and b is an integer greater than or equal to 1), and optionally a and b independently are less than or equal to 150 or less than or equal to 100. In examples, R₅ and R₇ are hydrogen and R₆ and R₈ are substituted or unsubstituted C₄ or greater alkenyl in the first portion and the second portion. In examples, at least one of R₅, R₆, R₇, and R₈ comprise acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or combinations thereof. In examples, at least one of R₁, R₂, R₃, and R₄ comprise acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or combinations thereof.

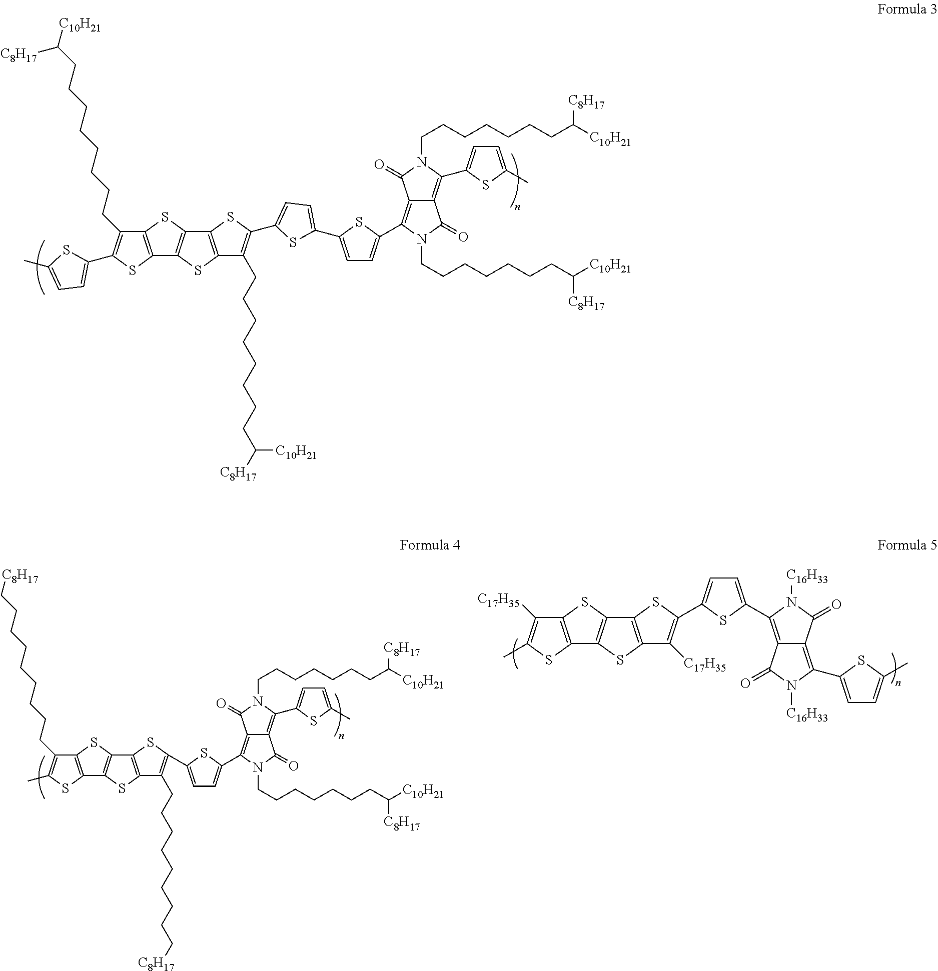

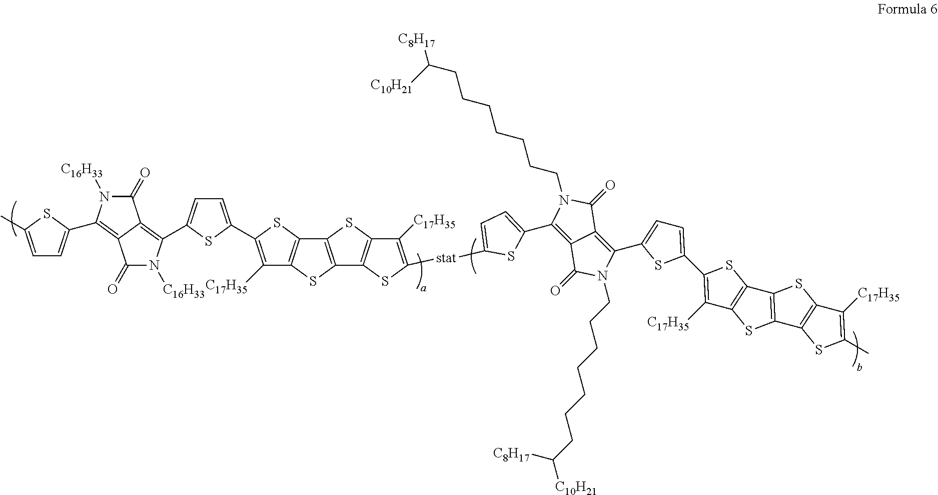

In some examples, the at least one UV-curable side chain comprises at least one alkyl chain terminated by a functional group which can be UV crosslinked by a [2+2]/[4+2] mechanism (e.g., cinnamates, coumarins and chalcones).

In examples, the OSC has a solubility of at least, or less than, any of: 0.5 mg/mL, 1 mg/mL, 2 mg/mL, 3 mg/mL, 4 mg/mL, 5 mg/mL, 10 mg/mL, 15 mg/mL, 20 mg/mL, 25 mg/mL, 30 mg/mL, 35 mg/mL, 40 mg/mL, or any value therein, or any range defined by any two of those endpoints. In examples, the OSC has a solubility of at least 1 mg/mL at room temperature.

In examples, the OSC has hole mobilities of at least, or less than, any of: 0.1 cm²V⁻¹s⁻¹, 0.25 cm²V⁻¹s⁻¹, 0.5 cm²V⁻¹s⁻¹, 0.75 cm²V⁻¹s⁻¹, 1 cm²V⁻¹s⁻¹, 2 cm²V⁻¹s⁻¹, 3 cm²V⁻¹s⁻¹, 4 cm²V⁻¹s⁻¹, 5 cm²V⁻¹s⁻¹, 10 cm²V⁻¹s⁻¹, 15 cm²V⁻¹s⁻¹, 20 cm²V⁻¹s⁻¹, 25 cm²V⁻¹s⁻¹, 30 cm²V⁻¹s⁻¹, 35 cm²V⁻¹s⁻¹, 40 cm²V⁻¹s⁻¹, or any value therein, or any range defined by any two of those endpoints. Hole mobilities may be equal to or greater than any of these values. In examples, the OSC has hole mobilities of 1 to 4 cm²V⁻¹s⁻¹. In examples, the OSC has hole mobilities of 2 cm²V⁻¹s⁻¹. In examples, the OSC has hole mobilities of 2 cm²V⁻¹s⁻¹ or more.

In examples, the OSC polymers have On/Off ratios of greater than 10⁴. In examples, the OSC polymers have On/Off ratios of greater than 10⁶.

In examples, the OSC polymers have a threshold voltage in thin film transistor devices of at least, or less than, any of: −20V, −15V, −10V, −5V, −4V, −3V, −2V, −1V, 0V, 1V, 2V, 3V, 4V, 5V, 10V, 15V, 20V, or any value therein or any range defined by any two of those endpoints. In examples, the OSC polymers have a threshold voltage in a range of 1 V to 3 V in thin film transistor devices. In examples, the OSC polymers have a threshold voltage of 2 V in thin film transistor devices.

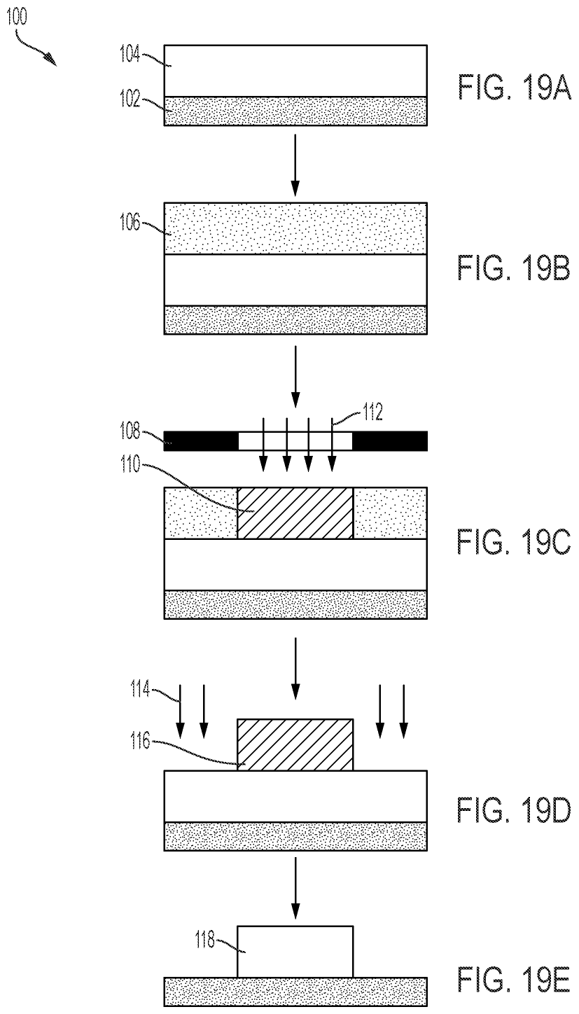

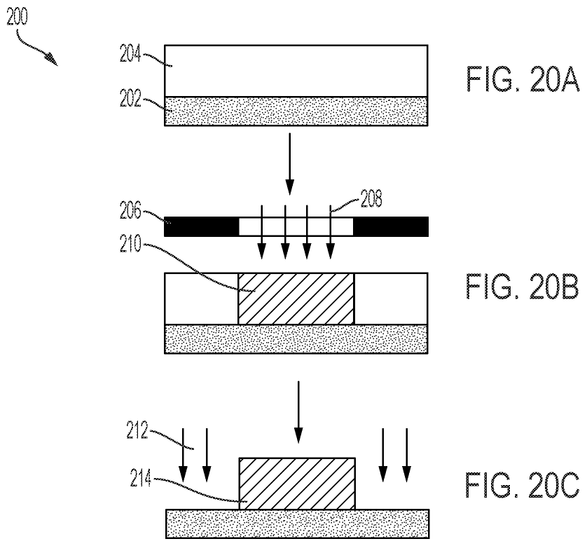

The OSC polymer disclosed herein (e.g., with at least one UV-curable side chain), enables direct UV crosslinking and patterning, thereby leading to improved patterning effects and OFET devices performance. For example, compared with conventional photolithography (described in FIGS. 19A-19E), directly UV curable cross-bred OSC polymers reduce the number of pattern processing steps to only two steps (e.g., FIGS. 20A-20C). Traditional processing steps, such as coating with compatible photoresists, etching the active material, and resist stripping become unnecessary due to the intrinsic UV patternability of the cross-bred OSC polymers disclosed herein. This reduction of manufacturing steps has a direct benefit in avoiding device performance degradation, since contact with potentially harmful solvents during resist coating and aggressive plasma etching atmospheres are avoided. Moreover, the reduction of steps may also significantly reduce manufacturing cost, equipment investment, as well as shorten the manufacturing cycle in OTFT manufacturing.

The disclosed cross-bred OSC polymers having the at least one UV-curable side chain have no phase separation issues and have stronger solvent resistance due to covalent-bond crosslinking. Thus, they are easier to process, leading to better reproducibility for solution processable OSC thin films. The chemical and physical properties of the cross-bred OSC polymers disclosed herein are also highly tunable by manipulating ratios among different monomers. The crosslinked OSC polymer networks formed using the disclosed cross-bred OSC polymers having the at least one UV-curable side chain help polymer chain alignment at elevated temperatures, offering higher temperature resistance of OTFT devices made thereof, as well as longer device life time and higher weatherability.

### Crosslinker

In examples, a polymer blend comprises at least one organic semiconductor (OSC) polymer and at least one crosslinker, such that the crosslinker includes at least one of: acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or a combination thereof. In examples, the at least one crosslinker comprises C═C bonds, thiols, oxetanes, halides, azides, or combinations thereof.

In examples, the crosslinker may be a small molecule or a polymer that reacts with the OSC polymer by one or a combination of reaction mechanisms, depending on functional moieties present in the crosslinker molecule. For example, crosslinkers comprising thiol groups may react with double bonds in the OSC polymer via thiol-ene click chemistry. In examples, crosslinkers comprising vinyl groups may react with double bonds in the OSC polymer via addition reaction. In examples, crosslinkers (comprising thiols, vinyl groups, etc., or combinations thereof) may react with crosslinkable functionalities incorporated in the side chains of OSC polymers. These include, for example, acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or combinations thereof.

In aspects, which is combinable with any of the other aspects or embodiments, the at least one crosslinker comprises at least one of, in which each n independently is an integer greater than or equal to 1:

TABLE 1

| | |
|---|---|
| 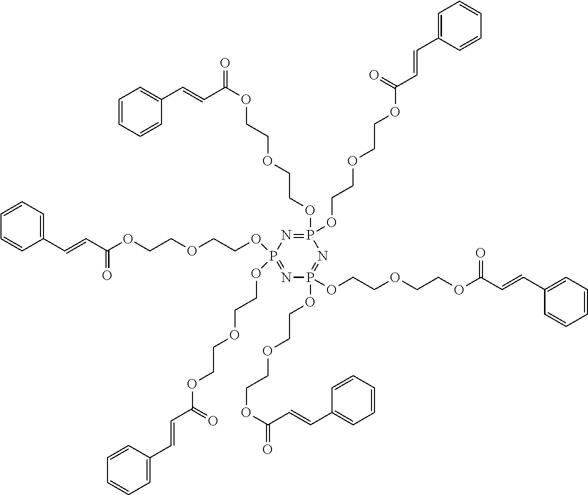 [SMILES: O=C(/C=C/c1ccccc1)OCCOCCOP1(OCCOCCOC(=O)/C=C/c2ccccc2)=NP(OCCOCCOC(=O)/C=C/c2ccccc2)(OCCOCCOC(=O)/C=C/c2ccccc2)=NP(OCCOCCOC(=O)/C=C/c2ccccc2)(OCCOCCOC(=O)/C=C/c2ccccc2)=N1] | C1 |
| 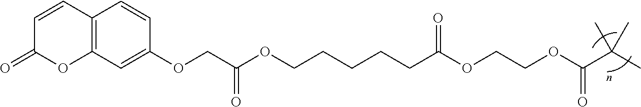 | C2 |
| 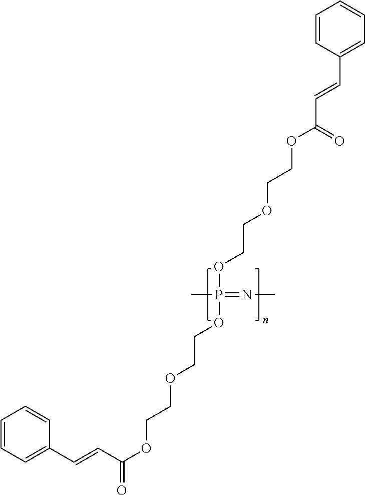 | C3 |
| 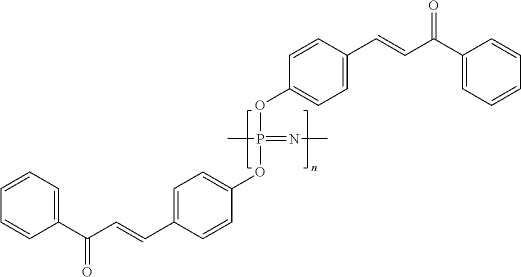 | C4 |
| 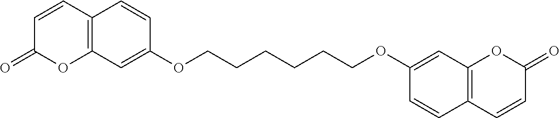 [SMILES: O=c1ccc2ccc(OCCCCCCOc3ccc4ccc(=O)oc4c3)cc2o1] | C5 |

### Photosensitizer

In examples, a polymer blend comprises at least one OSC polymer, at least one crosslinker, and at least one photosensitizer. In examples, a polymer blend comprises at least one OSC polymer and at least one photosensitizer.

Photosensitizers are molecules that enable a chemical change in another molecule in a photochemical process and which may be used in photo-polymerization, photo-crosslinking, and photo-degradation polymer chemistry reactions. Photosensitizers are also used to generate triplet excited states in organic molecules for use in photocatalysis, photon up-conversion and photodynamic therapy. Functionally, photosensitizers absorb ultraviolet (UV) or visible electromagnetic radiation and transfer that energy to potentially ionize adjacent molecules. Moreover, photosensitizers usually have large de-localized x systems, which lowers the energy of HOMO orbitals.

In examples, the at least one photosensitizer may include those with chemical structures shown in Table 2 below:

TABLE 2

| | |
|---|---|
| 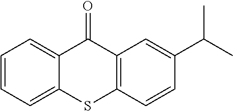 [SMILES: CC(C)c1ccc2sc3ccccc3c(=O)c2c1] | P1 |
| 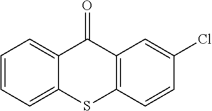 [SMILES: O=c1c2ccccc2sc2ccc(Cl)cc12] | P2 |
| 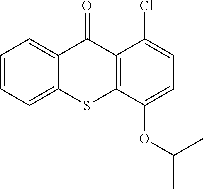 [SMILES: CC(C)Oc1ccc(Cl)c2c(=O)c3ccccc3sc12] | P3 |
| 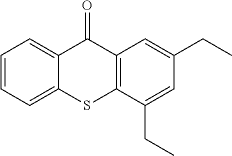 [SMILES: CCc1cc(CC)c2sc3ccccc3c(=O)c2c1] | P4 |
| 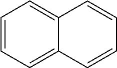 | P5 |
| 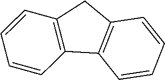 [SMILES: c1ccc2c(c1)Cc1ccccc1-2] | P6 |
| 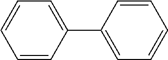 [SMILES: c1ccc(-c2ccccc2)cc1] | P7 |
| 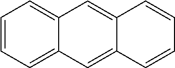 [SMILES: c1ccc2cc3ccccc3cc2c1] | P8 |
| 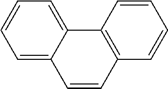 [SMILES: c1ccc2c(c1)ccc1ccccc12] | P9 |
| 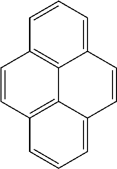 [SMILES: c1cc2ccc3cccc4ccc(c1)c2c34] | P10 |
| 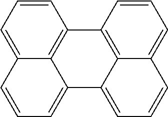 [SMILES: c1cc2cccc3c4cccc5cccc(c(c1)c23)c54] | P11 |
| 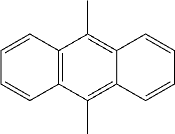 [SMILES: Cc1c2ccccc2c(C)c2ccccc12] | P12 |
| 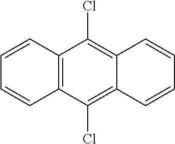 [SMILES: Clc1c2ccccc2c(Cl)c2ccccc12] | P13 |
| 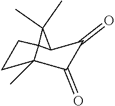 | P14 |

### Additives

In examples, a polymer blend comprises at least one OSC polymer, at least one crosslinker, at least one photosensitizer, and at least one additive, such as antioxidants (i.e., oxygen inhibitors), lubricants, compatibilizers, leveling agents, nucleating agents, or combinations thereof. In examples, oxygen inhibitors include phenols, thiols, amines, ethers, phosphites, organic phosphines, hydroxylamines, or combinations thereof. In examples, a polymer blend comprises at least one OSC polymer, at least one photosensitizer, and at least one additive.

### Polymer Blend

In examples, the performance of a device comprising the OSC polymer may be improved by blending the OSC polymer with a crosslinker. In examples, the OSC polymer is blended with a crosslinker in a solvent. In examples, the solvent is chloroform, methylethylketone, toluene, xylenes, chlorobenzene, 1,2-dichlorobenzene, 1,2,4-trichlorobenzene, tetralin, naphthalene, chloronaphthalene, or combinations thereof. In examples, a mixture of more than one solvent may be used.

In examples, the at least one OSC polymer is present at 1 wt. %, or 2 wt. %, or 3 wt. %, or 5 wt. %, or 10 wt. %, or 15 wt. %, or 20 wt. %, or 25 wt. %, or 30 wt. %, or 35 wt. %, or 40 wt. %, or 50 wt. %, or 60 wt. %, or 70 wt. %, or 80 wt. %, or 90 wt. %, or 95 wt. %, or 99 wt. %, or any range defined by any two of those endpoints. In examples, the at least one crosslinker is present at 0.1 wt. %, or 0.2 wt. %, or 0.3 wt. %, or 0.5 wt. %, or 0.8 wt. %, or 1 wt. %, or 2 wt. %, or 3 wt. %, or 5 wt. %, or 10 wt. %, or 15 wt. %, or 20 wt. %, or 25 wt. %, or 30 wt. %, or 35 wt. %, or 40 wt. %, or 45 wt. %, or 50 wt. %, or 55 wt. %, or 60 wt. %, or 65 wt. %, or 70 wt. %, or 75 wt. %, or 80 wt. %, or 85 wt. %, or 90 wt. %, or 95 wt. %, or 99 wt. %, or any range defined by any two of those endpoints. In examples, the at least one photosensitizer is present at 0.1 wt. %, or 0.2 wt. %, or 0.3 wt. %, or 0.4 wt. %, or 0.5 wt. %, or 0.6 wt. %, or 0.7 wt. %, or 0.8 wt. %, or 0.9 wt. %, or 1 wt. %, or 1.5 wt. %, or 2 wt. %, or 2.5 wt. %, or 3 wt. %, or 3.5 wt. %, or 4 wt. %, or 4.5 wt. %, or 5 wt. %, or 6 wt. %, or 7 wt. %, or 8 wt. %, or 9 wt. %, or 10 wt. %, or any range defined by any two of those endpoints. In examples, the at least one antioxidant, lubricant, compatibilizer, leveling agent, or nucleating agent may each be present, independently, at 0.05 wt. %, or 0.1 wt. %, or 0.2 wt. %, or 0.3 wt. %, or 0.4 wt. %, or 0.5 wt. %, or 0.6 wt. %, or 0.7 wt. %, or 0.8 wt. %, or 0.9 wt. %, or 1 wt. %, or 1.5 wt. %, or 2 wt. %, or 2.5 wt. %, or 3 wt. %, or 3.5 wt. %, or 4 wt. %, or 4.5 wt. %, or 5 wt. %, or any range defined by any two of those endpoints.

In examples, the blend consists of OSC polymers as described herein. In examples, the blend comprises at least two of: OSC polymers, crosslinkers, photosensitizers, and additives as described herein. In examples, the blend comprises at least three of: OSC polymers, crosslinkers, photosensitizers, and additives as described herein. In examples, the blend comprises all of: OSC polymers, crosslinkers, photosensitizers, and additives as described herein.

### OTFT Device Fabrication

Applications using OTFT devices require patterning of organic semiconducting materials to prevent undesired high off-currents and crosstalk between adjacent devices. As explained above, photolithography is a common patterning technique in semiconductor device fabrication. However, photolithography usually involves harsh O₂ plasma during pattern transfer or photoresist removal and aggressive developing solvents which may severely damage the OSC layer and lead to significant deterioration of OTFT device performance. In other words, conjugated organic materials tend to degrade when exposed to light and the chemicals used in photolithography may have an adverse effect on organic thin film transistors. Therefore, patterning of organic semiconducting materials using photolithography is not practical.

FIGS. 19A-19E illustrate traditional patterning techniques 100 of organic semiconductor blends utilizing photoresists. In a first step (FIG. 19A), a thin film 104 of the blended OSC polymer is deposited over a substrate 102 followed by deposition of a photoresist layer 106 thereon in FIG. 19B. Optionally, the thin film 104 may be thermally annealed. The photoresist deposition may be conducted using processes known in the art such as spin coating. For example, the photoresist, rendered into a liquid form by dissolving the solid components in a solvent, is poured onto the substrate, which is then spun on a turntable at a high speed producing the desired film. Thereafter, the resulting resist film may experience a post-apply bake process (i.e., soft-bake or prebake) to dry the photoresist in removing excess solvent.

In the step of FIG. 19C, the photoresist layer 106 is exposed to UV light 112 through a master pattern called a photomask 108 positioned some distance away from the photoresist layer 106 to form a higher crosslinked portion 110 of the photoresist layer 106. The exposure to UV light operates to change the solubility of the photoresist in a subsequent developer solvent solution for pattern formation atop the substrate. Prior to the developer, the resist layer may experience a post exposure bake. In the step of FIG. 19D, the pattern 116 of the photoresist layer is transferred into the thin film 104 via subtractive etching 114 (i.e., O₂ plasma dry etching). The patterned photoresist layer 116 “resists” the etching and protects the material covered by the photoresist. When the etching is complete, the photoresist is stripped (e.g., using organic or inorganic solutions, and dry (plasma) stripping) leaving the desired pattern 118 etched into the thin film layer.

However, as explained above, aspects of traditional photolithography processes such as harsh O₂ plasma during pattern transfer and aggressive photoresist developer solvents and/or stripping solvents may severely damage the OSC layer and lead to significant deterioration of device performance.

FIGS. 20A-20C illustrate patterning techniques 200 of organic semiconductor blends, according to embodiments. In a first step (FIG. 20A), a thin film 204 of the blended OSC polymer is deposited over a substrate 202. Optionally, the thin film 204 may be thermally annealed. In examples, depositing comprises at least one of spin coating; dip coating; spray coating; electrodeposition; meniscus coating; plasma deposition; and roller, curtain and extrusion coating. The thin film 204 was prepared as a polymer blend described above comprising at least one organic semiconductor (OSC) polymer, and optionally, at least one crosslinker, at least one photosensitizer, and at least one additive.

In examples, the blending includes dissolving the at least one OSC polymer in a first organic solvent to form a first solution, dissolving the at least one crosslinker in a second organic solvent to form a second solution, and dissolving at least one photosensitizer in a third organic solvent to form a third solution; and combining the first, second, and third solutions in any suitable order to create the polymer blend. In examples, the first, second, and third solutions may be combined simultaneously. In examples, the at least one OSC polymer, at least one crosslinker, and at least one photosensitizer may be prepared together in a single organic solvent. The weight compositions of each component of the polymer blend is as provided above.

In examples, after the thin film of the blended OSC polymer is deposited over the substrate and before exposing the thin film to UV light, the thin film may be heated at a temperature in a range of 50° C. to 200° C. for a time in a range of 10 sec to 10 min to remove excess solvent.

In a second step (FIG. 20B), the thin film 204 was exposed to UV light 208 through a photomask 206 to form a higher crosslinked portion 210 of the thin film 204. In examples, the exposing comprises exposing the thin film to UV light having an energy in a range of 10 mJ/cm² to 600 mJ/cm² (e.g., 400 mJ/cm²) for a time in a range of 1 sec to 60 sec (e.g., 10 sec). In examples, the UV light may have an energy in a range of 300 mJ/cm² to 500 mJ/cm² and be operable for a time in a range of 5 sec to 20 sec. Similar to photoresist functionality described in FIGS. 19A-19E, the exposure to UV light operates to change the solubility of the thin film in a subsequent developer solvent solution for pattern formation atop the substrate.

In the step of FIG. 20C, when light exposure is complete, the portion of the thin film 204 not exposed to UV light 208 was stripped using a predetermined solvent 212, thereby leaving the desired pattern 214 into the thin film layer. In other words, the higher crosslinked portion 210 was developed in a solvent to remove an un-patterned region of the thin film 204. In examples, the developing comprises exposing the un-patterned region of the thin film to a solvent comprising chlorobenzene, 1,2-dichlorobenzene, 1,3-dichlorobenzene, 1,2,4-trichlorobenzene, dioxane, p-xylene, m-xylene, toluene, cyclopentanone, cyclohexanone, methyl lactate, 2-butanone, 2-pentanone, 3-pentanone, 2-heptanone, 3-heptanone, anisole, mesitylene, decalin, butylbenzene, cyclooctane, tetralin, chloroform, or combinations thereof, for a time in a range of 10 sec to 10 min. In examples, the developer solution comprises chlorobenzene, p-xylene, dioxane, or combinations thereof.

In examples, after developing the patterned thin film in a solvent to remove the un-patterned region of the thin film, the thin film may be heated at a temperature in a range of 50° C. to 200° C. for a time in a range of 10 sec to 30 min.

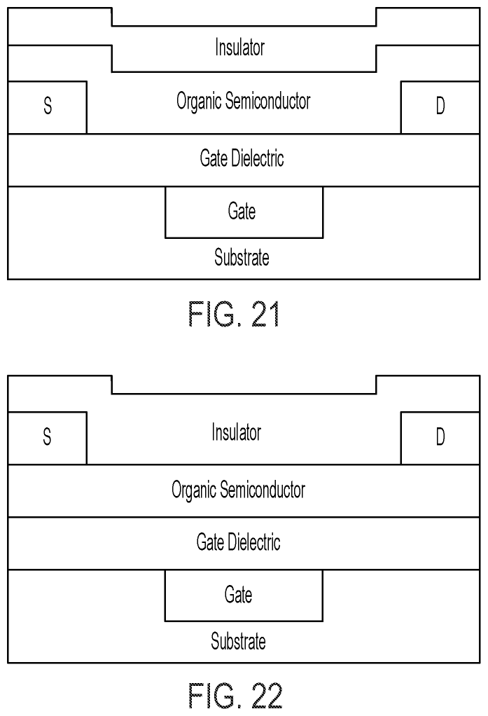

Thereafter, the OTFT devices may be completed by forming a gate electrode over the substrate; forming a gate dielectric layer over the substrate; forming patterned source and drain electrodes over the gate dielectric layer; forming an organic semiconductor active layer over the and gate dielectric layer, and forming an insulator layer over the patterned organic semiconductor active layer. (FIGS. 21 and 22).

Various aspects are contemplated herein, several of which are set forth in the paragraphs below. It is explicitly contemplated that any aspect or portion thereof can be combined to form a combination.

Aspect 1. A semiconductor device, comprising:
at least one organic semiconductor (OSC) polymer and at least one photosensitizer,
wherein the at least one OSC polymer is a diketopyrrolopyrrole-fused thiophene polymeric material, wherein the fused thiophene is beta-substituted.

Aspect 2. The semiconductor device of aspect 1, or any preceding aspect, wherein the at least one OSC polymer comprises a first OSC polymer and a second OSC polymer.

Aspect 3. The semiconductor device of aspect 2, or any preceding aspect, wherein the first OSC polymer and the second OSC polymer have identical conjugated backbones.

Aspect 4. The semiconductor device of aspect 2, or any preceding aspect, wherein a weight ratio between the first OSC polymer and the second OSC polymer ranges from 4:1 to 1:4.

Aspect 5. The semiconductor device of any one of aspects 1-4, or any preceding aspect, comprising an isotropic charge mobility of at least 0.40 cm² V⁻¹ s⁻¹.

Aspect 6. The semiconductor device of any one of aspects 1-5, or any preceding aspect, comprising a bottom-gate bottom-contact (BGBC)-configurated organic thin film transistor (OTFT) array.

Aspect 7. The semiconductor device of any one of aspects 1-6, or any preceding aspect, wherein the at least one OSC polymer comprises a repeat unit of Formula 1, Formula 2, a salt thereof, or any combination thereof:

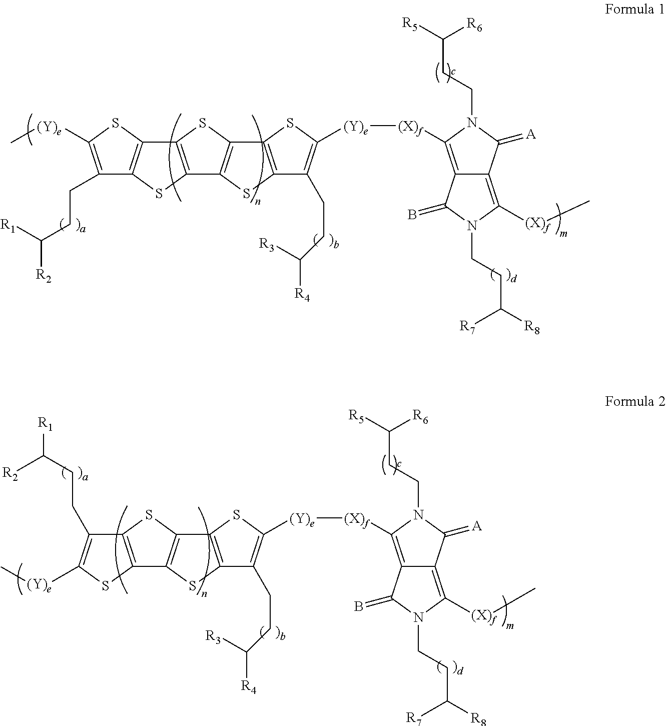

wherein:
m is an integer greater than or equal to one;
n is 0, 1, or 2;
R₁, R₂, R₃, R₄, R₅, R₆, R₇, and R₈ independently are hydrogen, substituted or unsubstituted C₄ or greater alkyl, substituted or unsubstituted C₄ or greater alkenyl, substituted or unsubstituted C₄ or greater alkynyl, or C₅ or greater cycloalkyl;
a, b, c, and d independently are integers greater than or equal to 3;
e and f independently are integers greater than or equal to zero;
X and Y independently are a covalent bond, an optionally substituted aryl group, an optionally substituted heteroaryl, an optionally substituted fused aryl or fused heteroaryl group, an alkyne or an alkene; and
A and B independently are either S or O;
with the provisos that:
   (i) at least one of R₁ or R₂; one of R₃ or R₄; one of R₅ or R₆; and one of R₇ or R₈ is a substituted or unsubstituted alkyl, substituted or unsubstituted alkenyl, substituted or unsubstituted alkynyl, or cycloalkyl;
   (ii) if any of R₁, R₂, R₃, or R₄ is hydrogen, then none of R₅, R₆, R₇, or R₈ are hydrogen;
   (iii) if any of R₅, R₆, R₇, or R₈ is hydrogen, then none of R₁, R₂, R₃, or R₄ are hydrogen;
   (iv) e and f cannot both be 0;
   (v) if either e or f is 0, then c and d, independently, are integers greater than or equal to 5; and
   (iv) an OSC polymer of the at least one OSC polymer has a molecular weight,
wherein the molecular weight of the OSC polymer is greater than 10,000.

Aspect 8. The semiconductor device of aspect 7, or any preceding aspect, wherein at least one of R₁, R₂, R₃, and R₄ comprise acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or combinations thereof.

Aspect 9. The semiconductor device of any one of aspects 1-8, or any preceding aspect, wherein the at least one OSC polymer comprises a first portion and a second portion, and at least one of the first portion or the second portion comprises at least one UV-curable side chain.

Aspect 10. The semiconductor device of aspect 9, or any preceding aspect, wherein the first portion comprises the at least one UV-curable side chain, the second portion comprises a repeat unit of Formulas 3-6 or a salt thereof:

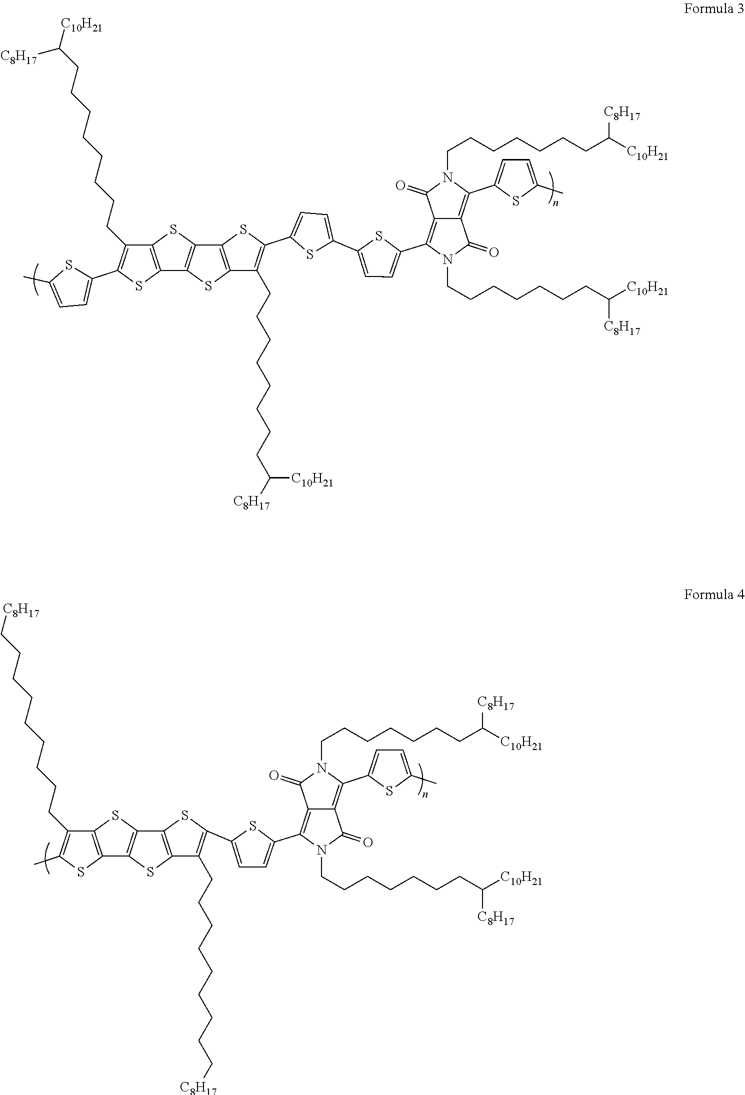

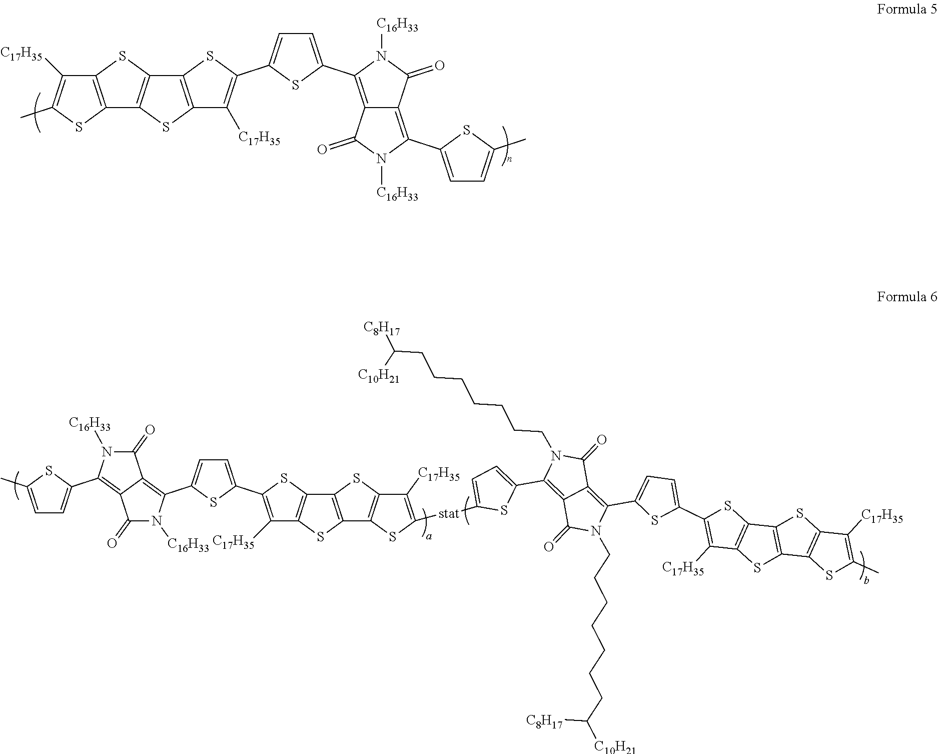

wherein:
each n in Formulas 3-5 independently is an integer greater than or equal to 1; and
a and b in Formula 6 independently are an integer greater than or equal to 1.

Aspect 11. The semiconductor device of any one of aspects 1-8, or any preceding aspect, wherein the at least one OSC polymer comprises a first portion and a second portion, wherein the first portion comprises at least one UV-curable side chain, and the second portion comprises a repeat unit of Formulas 3-6 or a salt thereof.

Aspect 12. The semiconductor device of any one of aspects 9-11, or any preceding aspect, wherein the at least one UV-curable side chain comprises at least one alkyl chain terminated by a functional group which can be UV crosslinked by a [2+2]/[4+2] mechanism.

Aspect 13. The semiconductor device of any one of aspects 1-8, or any preceding aspect, wherein the at least one OSC polymer comprises a first portion and a second portion, wherein the first portion comprises R₅ and R₇ are hydrogen and R₆ and R₈ are substituted or unsubstituted C₄ or greater alkenyl, and the second portion comprises a repeat unit of Formulas 3-6 or a salt thereof.

Aspect 14. The semiconductor device of any one of aspects 1-8, or any preceding aspect, wherein the at least one OSC polymer comprises a first portion and a second portion, wherein R₅ and R₇ are hydrogen and R₆ and R₈ are substituted or unsubstituted C₄ or greater alkenyl in the first portion and the second portion.

Aspect 15. The semiconductor device of any one of aspects 1-14, or any preceding aspect, wherein the at least one OSC polymer comprises at least one of:
a solubility of at least 0.5 mg/mL at room temperature;
hole mobility 0.1 cm²V⁻¹s⁻¹;
an on/off ratio of greater than 10⁴;
a threshold voltage in thin film transistor devices of at least −20 V.

Aspect 16. The semiconductor device of any one of aspects 1-15, or any preceding aspect, wherein the at least one OSC polymer is in a polymer blend further comprising at least one crosslinker.

Aspect 17. The semiconductor of aspect 16, or any preceding aspect, wherein the at least one crosslinker comprises at least one of:

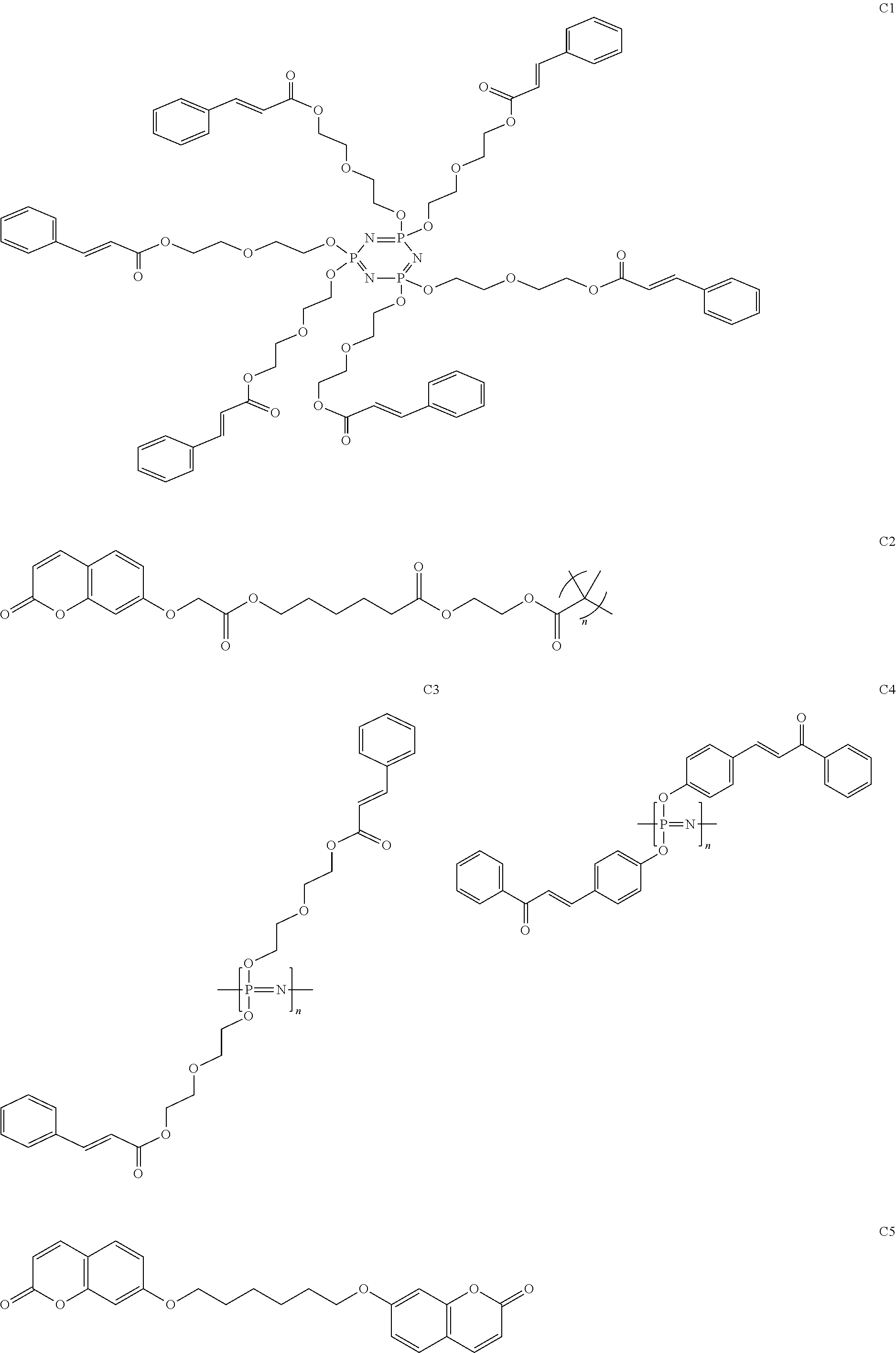

wherein each n independently is an integer greater than or equal to 1.

Aspect 18. The semiconductor of aspect 16 or aspect 17, or any preceding aspect, wherein the polymer blend further comprises at least one photosensitizer.

Aspect 19. The semiconductor of aspect 18, or any preceding aspect, wherein the at least one photosensitizer comprises at least one of:

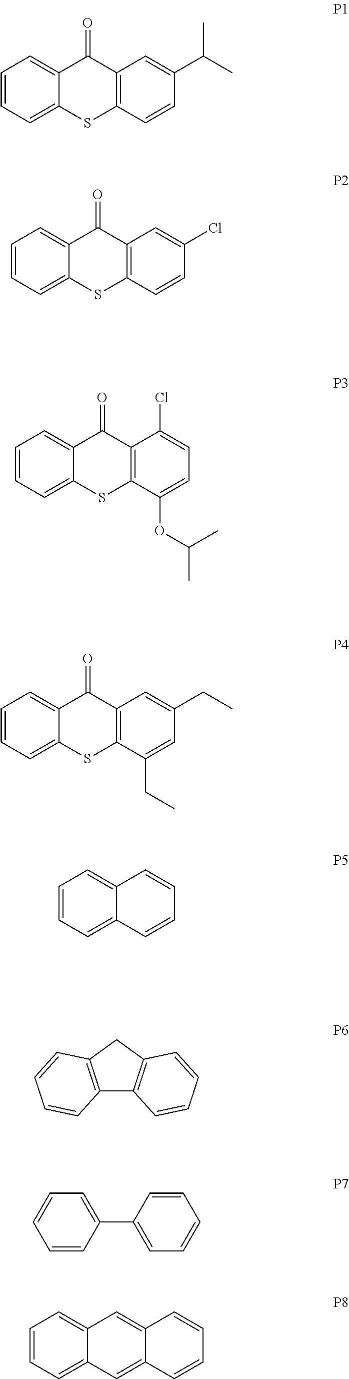

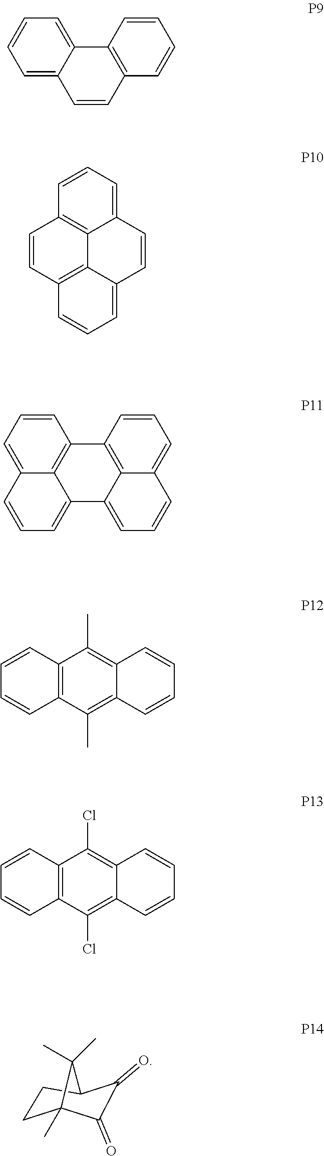

Aspect 20. A method of making the semiconductor device of any one of aspects 1-19, or any preceding aspect, the method comprising:
depositing the at least one OSC polymer on a substrate;
depositing a photoresist layer on the at least one OSC polymer;
optionally thermally annealing;
exposing the photoresist layer to UV light through a photomask to form an exposed photoresist layer;
optionally thermally baking; and
etching the exposed photoresist layer.

Aspect 21. A method of making the semiconductor device of any one of aspects 1-19, or any preceding aspect, the method comprising:
depositing the at least one OSC polymer on a substrate;
optionally thermally annealing;
exposing the photoresist layer to UV light through a photomask to form an exposed photoresist layer; and
subjecting the exposed photoresist layer to a solvent to remove un-patterned regions.

Aspect 22: A combination of any two or more of aspects 1-21, or any one or more portions thereof.

### EXAMPLES

The embodiments described herein will be further clarified by the following examples.

All experimental operations are done in a fume hood unless otherwise stated.

### Example 1—Composition of SP-2

SP-2 comprises at least two semiconducting polymers PTDPPTFT4-0C and PTDPPTFT4-10C, and a photosensitizer (Irgacure ITX, BASF). In examples, the photosensitizer is P1 from Table 2 (CAS No. 5495-84-1). PTDPPTFT4-0C and PTDPPTFT4-10C are dissolved in chlorobenzene (CB) at a weight ratio of 1:1 to obtain a pseudo-homogeneous blending solution with a polymer concentration of 10 mg ml⁻¹. Before photolithography, ITX (3 wt. % of semiconducting polymers) is added to the above blending solution.

A first control sample comprises PTDPPTFT4-5C and 3 wt. % photosensitizer.

A second control sample is a modified SP-1 polymer comprising PTDPPTFT4-0C and an acrylate crosslinker (tris[2-(acryloyloxy)ethyl]isocyanurate) at a weight ratio of 1:1.

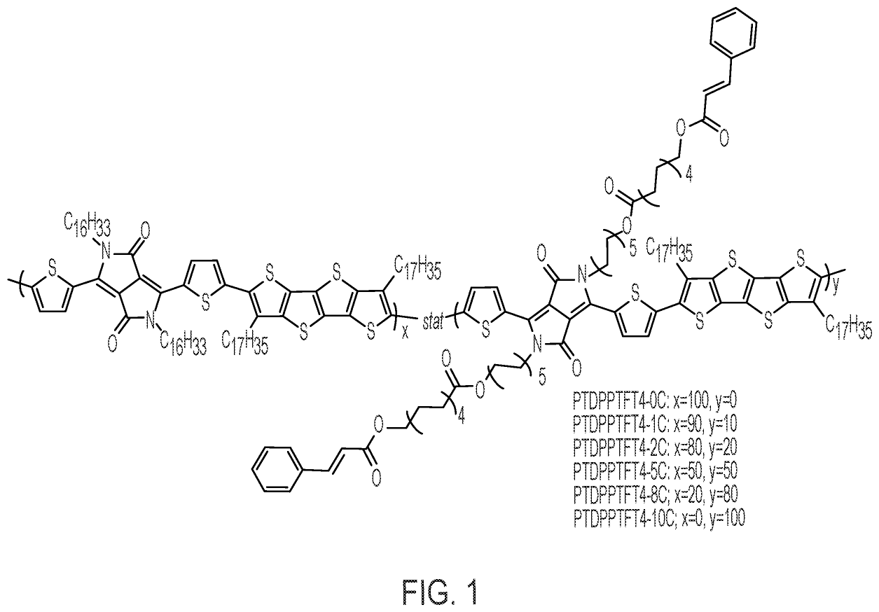

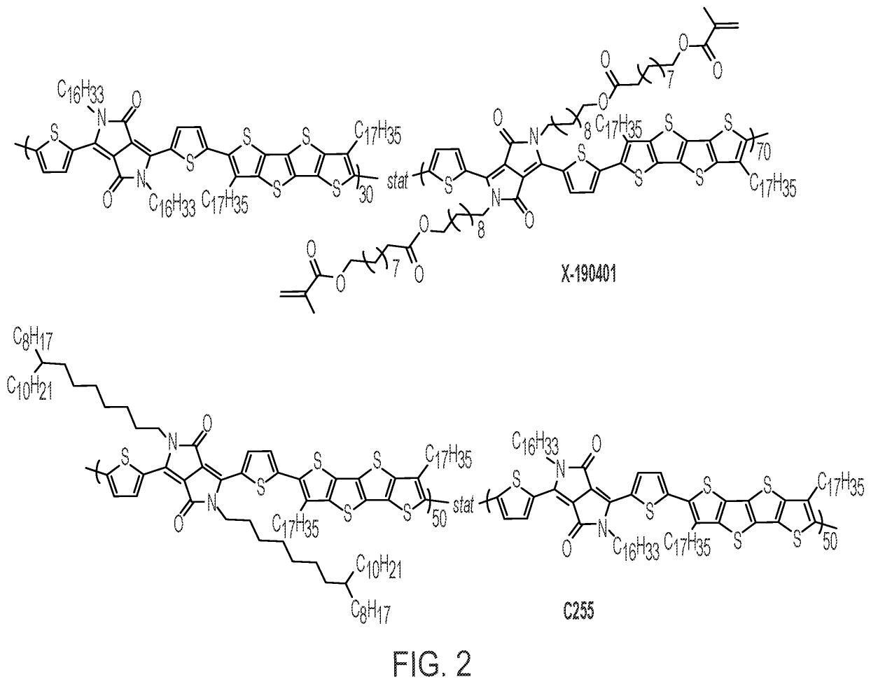

FIG. 1 illustrates polymer structures of semiconducting polymers PTDPPTFT4-zC, where x and y are relative mole ratio of DPP monomers, depicting SP-2, first control, and second control.

### Example 2—Fabrication of OTFTs

An n-type heavily doped Si wafer with a 300 nm SiO₂ layer (specific capacitance COX=11 nF cm⁻²) serves as bottom gate electrode and dielectric layer, respectively. To fabricate BGBC structured OTFTs, 5/60 nm-thick Cr/Ag layers are thermally evaporated on the pristine SiO₂/Si substrates through photolithography process (using S1813 photoresist) as bottom S/D electrodes. After lift-off by stripper (Remover PG, MicroChem), the substrate with S/D electrodes is rinsed with deionized (DI) water and ethanol, and then treated with an air plasma for 30 sec at a power of 100 W. As-cleaned substrate is treated with octadecyltrichlorosilane (OTS) in a vacuum oven at a temperature of 120° C., forming an OTS self-assembled monolayer, then sonicated in heptane, ethanol, and chloroform successively to remove redundant OTS molecules. Ultimately, 10 mg ml⁻¹ SP-2 solution is deposited on the as-treated substrate by spin-coating.

Photolithography is achieved via Microwriter ML3 laser direct writing photoengraving machine (Durham Magneto Optics Ltd.). The simplified photolithography process for SP-2 is described as following: spin-cast film is pre-baked at 130° C. for 2 min, then exposured by a mask aligner (385 nm UV light source) with a predetermined exposure dose (1200˜2800 mJ cm⁻²). Subsequently, the as-exposed film is soaked in chlorobenzene (CB) for 30 sec while shaking. Finally, a post-bake at 170° C. for 10 min is carried out to complete photolithography. To fabricate BGTC structured OTFT, a similar processed is used, with the difference being that the semiconducting layer was fabricated prior to S/D electrodes.

### Example 3—OTFT Arrays and Circuits

To fabricate buried gate electrodes, SiO₂/Si wafers coated with patterned photoresist S1813 (Microposit) is etched by 10% HF for 20 sec prior to metal deposition (5/25 nm Cr/Au). A photocrosslinkable acrylate resin is used as an organic dielectric, which comprises 77 wt. % ethyl acrylate (EA), 10 wt. % methacrylic acid 2-(ethylsulfonyl)ethyl ester, 10 wt. % tris[2-(acryloyloxy)ethyl]isocyanurate, 1 wt. % thiol additive [trimethylolpropane tris(3-mercaptopropionate)], and 2 wt. % photoinitiator [diphenyl(2,4,6-trimethylbenzoyl)phosphine oxide (TPO)].

Acrylate resin is deposited on the gate electrode via spin-coating with a rotation speed of 3000 rpm for 30 sec, followed by a pre-bake (80° C., 2 min) to stabilize the quality of film. After pre-bake, the film is exposed to 385-nm UV light with a dose of 1200 mJ cm⁻² and the patterns are obtained by developing at propylene glycol methyl ether acetate (PGMEA) for 15 sec. Subsequently, 5/60 nm-thick Cr/Ag S/D electrodes are patterned through photolithography process and thermal evaporation. The semiconducting channel is patterned using SP-2/50 via simplified photolithography process described above. Except for thermal evaporation, all processes, including photolithography, annealing, and spin coating, are performed under an ambient atmosphere.

### Example 4—UV Patterning of Methacrylate-Functionalized Semiconducting Polymer X-190401 and Alkyl Side Chain Semiconducting Polymer C255

X-190401 and C255 (weight ratio of 1:1) are dissolved in chlorobenzene at a concentration of 10 mg/mL. The mixed solution is stirred at 60° C. overnight. 3 wt. % photoinitiator TPO is added to the solution at room temperature. After mixing, the solution is spin-coated on glass at a suitable speed for a predetermined time. Then, the coating composed of C255 and X-190401 is pre-baked at 80° C. for 2 min, followed by UV exposure at 365 nm UV mercury with 400 mJ/cm². Then, the coating is developed in mesitylene for 30 sec, and thereafter is dried by air-gun immediately.

### Example 5—Characterization

Sp-2 Demonstrates Better Patterning Performance than the UV Crosslinkable Cinnamate Functionalized Semiconducting Polymer

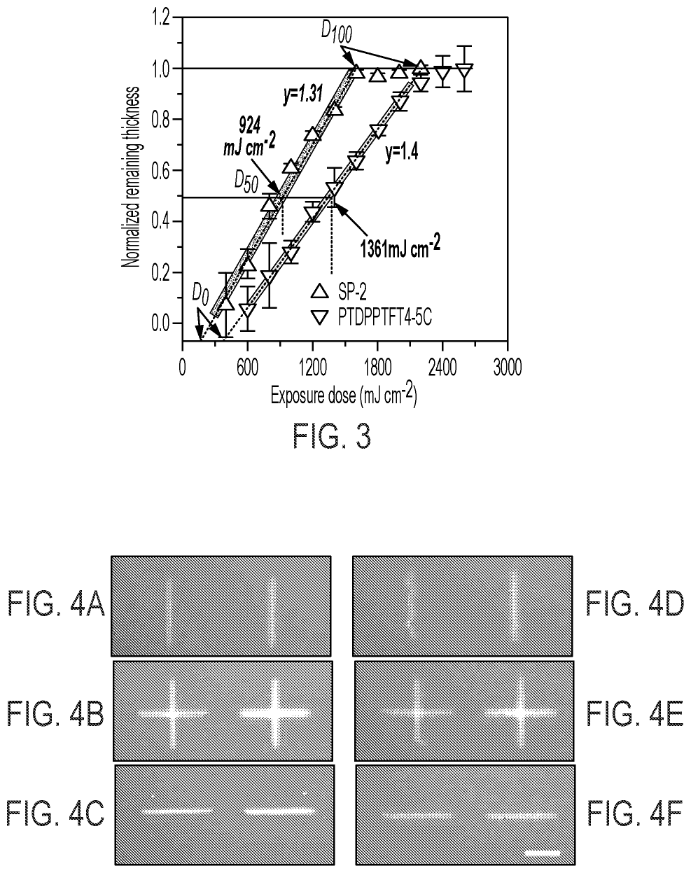

Sensitivity (S) and contrast (γ) of SP-2 was evaluated based on solubility curves of FIG. 3. FIG. 3 illustrates contrast curves of SP-2 and PTDPPTFT4-5C. γ is calculated by eq. (1):

$$γ = {[ log ( {D}_{100} / {D}_{0} ) ]}^{- 1} \qquad ( 1 )$$

where D₁₀₀ and D₀ correspond to exposure doses for complete reaction (D₁₀₀) and initial reaction (D₀), respectively. Contrast is the rate at which solubility of chemical substances change before and after exposure of the photoresist material. D₅₀ is defined as sensitivity. Contrast (γ) is the value calculated using eq. (1) and plotted in FIG. 3. As shown in FIG. 3, SP-2 and PTDPPTFT4-5C have typical characters of negative tone photoresists. Film thickness increases in good linearity as exposure dose increases. The γ of PTDPPTFT4-5C (up to 1.40) and SP-2 (up to 1.31) are greater than 1. SP-2 has smaller sensitivity (924 mJ cm⁻²) compared with PTDPPTFT4-5C (1361 mJ cm⁻²). In FIG. 3, sensitivity is the x-axis value (exposure dose) corresponding to a y-axis value (relative residual thickness) equal to 0.5.

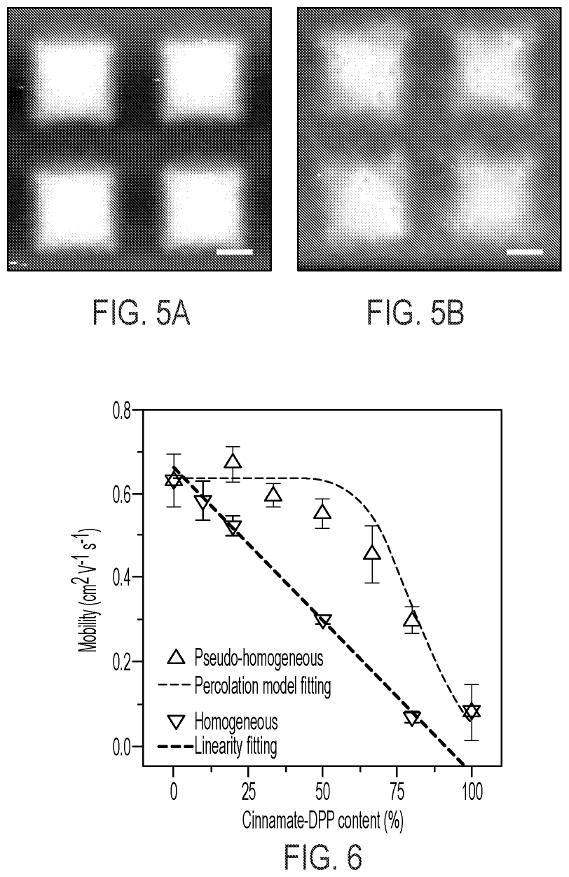

FIGS. 4A-4C illustrate OM images of line-like patterns of SP-2 and FIGS. 4D-4F illustrate OM images of line-like patterns of PTDPPTFT4-5C (scale bar is 5 μm). FIG. 5A illustrates AFM images of square-like patterns of SP-2 and FIG. 5B illustrates AFM images of square-like patterns of PTDPPTFT4-5C (scale bar is 2.5 μm). FIGS. 4A-4C and 5A demonstrate outstanding photolithographic performance of SP-2 for line- and square-like patterns. The pattern edges were sharp and the statistical minimum line width was down to 0.8 μm, closing to the resolution limit of the mask aligner. For PTDPPTFT4-5C, the line patterns in the vertical direction were observably deformed after development (FIGS. 4D-4F), and the square array patterns were found with inhomogeneous surface and blurry edges (FIG. 5B).

### Sp-2 Comprised of 1:1 Weight Ratio of PTDPPTFT4-0C and PTDPPTFT4-10C Exhibits the Optimal Balance Between Mobility and Patterning Performance

FIG. 6 illustrates the influence of cinnamate-DPP ratios on charge mobilities of SP-2 (green) and PTDPPTFT4-zC (red).

The electrical performance of SP-2 was evaluated by bottom-gate, bottom-contact (BGBC) structured OTFT devices. To improve device performance, a SiO₂/Si wafer was modified with self-assembled monolayers of octadecyltrichlorosilane (OTS). For UV crosslinkable semiconducting polymer PTDPPTFT4-zC, with increasing ratio of cinnamate-DPP, the mobility declined from 0.63 cm² V⁻¹ s⁻¹ to 0.09 cm² V⁻¹ s⁻¹ in linearity (FIG. 6). PTDPPTFT4-5C (˜0.3 cm² V⁻¹ s⁻¹) remained about 50% mobility of PTDPPTFT4-0C.

In comparison, five pseudo-homogeneous semiconducting photoresists SP-2 were prepared with weight ratios of PTDPPTFT4-0C and PTDPPTFT4-10C being 4:1, 2:1, 1:1, 1:2 and 1:4, respectively. The mobilities of SP-2 declined only slightly till the point of 1:1 weight ratio (i.e., 50% cinnamate-DPP content), where the average mobility of SP-2 was 0.55 cm² V⁻¹ s⁻¹, equivalent of 88.7% mobility of PTDPPTFT4-0C.

As the weight ratio of PTDPPTFT4-10C further increased, mobility dropped rapidly. Notably, SP-2 with 1:1 weight ratio of PTDPPTFT4-0C and PTDPPTFT4-10C exhibited the optimal balance between mobility and patterning performance, as SP-2 containing less PTDPPTFT4-10C were found to easily leave residues on the substrate after development, while higher PTDPPTFT4-10C ratios resulted in rapidly deteriorated charge mobilities. In the following description, SP-2/50 specifically refers to the blend comprised of 1:1 weight ratio of PTDPPTFT4-0C and PTDPPTFT4-10C.

### Sp-2 Exhibits Outstanding Processing and Environmental Stability

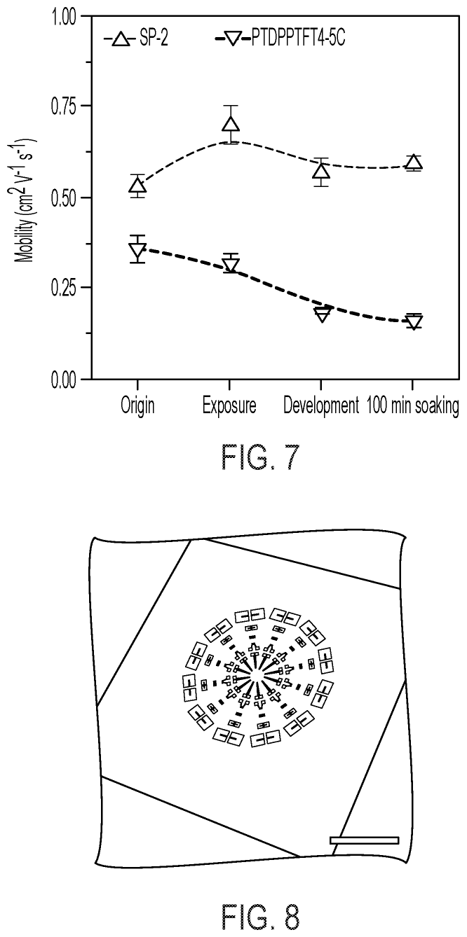

FIG. 7 illustrates processing and environmental stabilities of SP-2/50 and PTDPPTFT4-5C. Mobility of SP-2 increases from 0.53 cm² V⁻¹ s⁻¹ to 0.72 cm² V⁻¹ s⁻¹ after UV exposure, then declining to 0.55 cm² V⁻¹ s⁻¹ after development (soaking in chlorobenzene for 30 sec) and remained almost unchanged after 100 minutes soaking in the developer. In comparison, mobility of PTDPPTFT4-5C gradually declines from 0.340 to 0.305, 0.173, and 0.151 cm² V⁻¹ s⁻¹ throughout the photolithography processes, showing a remarkable drop of 53%.

### Charge Mobility of SP-2 was Independent of Channel Width

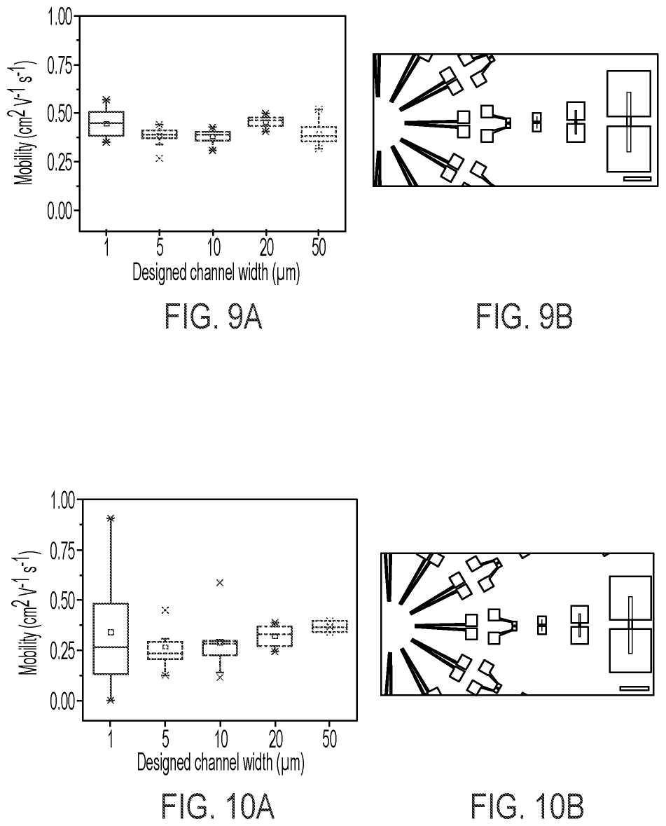

FIG. 8 illustrates, from the center to the periphery, five (5) concentric circular OTFT arrays with increasing channel widths of 1, 5, 10, 20, and 50 μm, respectively (scale bar is 5 mm). Each array comprises twelve (12) narrow-channel devices with a width-to-length ratio of 1:3. It was determined that as channel width decreases, average mobility remained almost unchanged (˜0.4 cm² V⁻¹ s⁻¹). FIG. 9A illustrates charge mobilities and FIG. 9B illustrates an optical microscopy (OM) image of SP-2/50 at various channel widths with channel direction perpendicular to the centrifugal force (scale bar is 500 μm). Mobility distributions were still narrow (i.e., in comparison with SP-1 in FIG. 10A) even for the smallest channel width of 1 μm (FIG. 9A). FIG. 10A illustrates charge mobilities and FIG. 10B illustrates an OM image of SP-1 at various channel widths with channel direction perpendicular to the centrifugal force (scale bar is 500 μm). By contrast, SP-1's mobilities decreased gradually as the channel width became smaller, accompanied by progressively widened mobility distributions (FIG. 10A). Especially in the case of the 1 μm channel width, devices showed huge mobility variations with relatively high fail rate of 16.7%.

### Charge Mobility of SP-2 was Independent of Centrifugal Directions

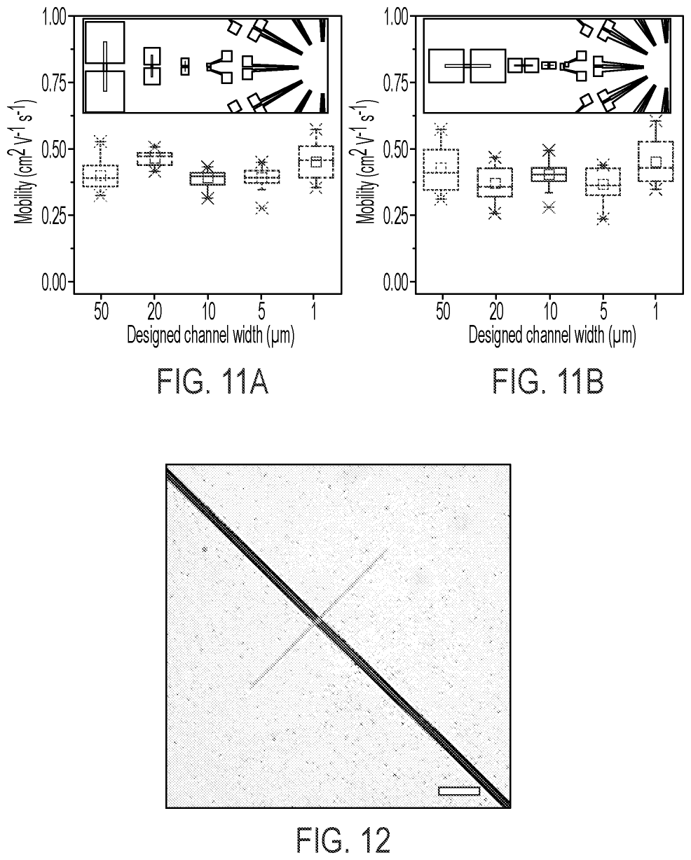

FIG. 11A illustrates charge mobilities of SP-2/50 at various channel widths with channel direction perpendicular to the centrifugal force and FIG. 11B illustrates charge mobilities of SP-2/50 at various channel widths with channel direction parallel to the centrifugal force. As shown in FIGS. 11A and 11B, charge mobilities of SP-2/50 were not sensitive to channel directions, e.g. devices with channel direction either perpendicular or parallel to the centrifugal force exhibited very similar charge mobilities and narrow distributions, indicating the isotropic short-range aggregation is the dominating molecular ordering mode for SP-2. The channel direction is the carrier transport direction, from source to drain. PTDPPTFT4-based semiconductor polymers are usually semicrystalline structures. Here, SP-2/50C has lower crystalline with smaller domain size, corresponding to a short-range aggregation. During deposition, these small grains are randomly oriented, forming isotropic short-range aggregation. In addition to the above crystalline regions, semiconductor thin films also have amorphous regions. Therefore, the isotropic short-range aggregation is the dominating molecular ordering mode for SP-2.

FIG. 12 demonstrated very good patterning resolution down to sub-micron scale.

### Uniform and Pseudo-Homogeneous Morphology of SP-2

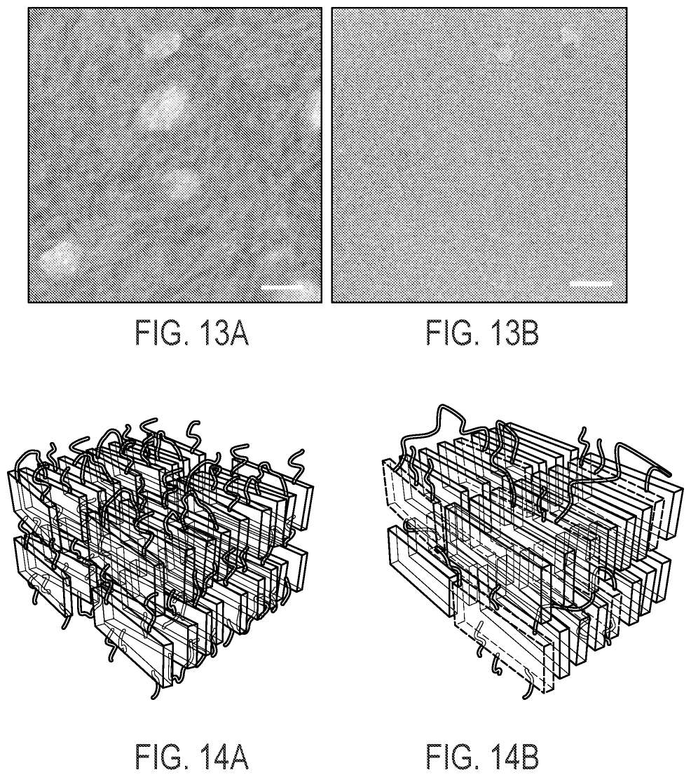

FIGS. 13A and 13B illustrate transmission electron microscopy (TEM) images of PTDPPTFT4-5C (FIG. 13A) and SP-2/50 (FIG. 13B) (scale bar is 100 nm). The figures clearly demonstrate that the developed PTDPPTFT4-5C has bundle-like aggregates and defects as large as 100 nm while developed SP-2/50 featured a uniform, ordered, and amorphous structure. Defects confine the movement of carriers, resulting in shifts in threshold voltage and decreases in carrier mobility. Among them, mobility is the most critical device performance indicator. Lack of defects or ordered structure is beneficial to carriers' transport, giving rise to higher mobility.

FIGS. 14A and 14B illustrate aggregation structures of PTDPPTFT4-5C (FIG. 14A) and SP-2/50 (FIG. 14B). Crosslinked side chains of PTDPPTFT4-5C severely disrupt the ordered stacking of conjugated backbones between adjacent molecules. SP-2/50 has a cross-linked phase and a conductive phase, with the cross-linked phase having no effect on the conductive phase's aggregation. As a result, SP-2/50 keeps ordered aggregation in small areas.

Down-Scaled Fabrication of Organic Devices with Improved Electrical Performance and Lower Power Consumption

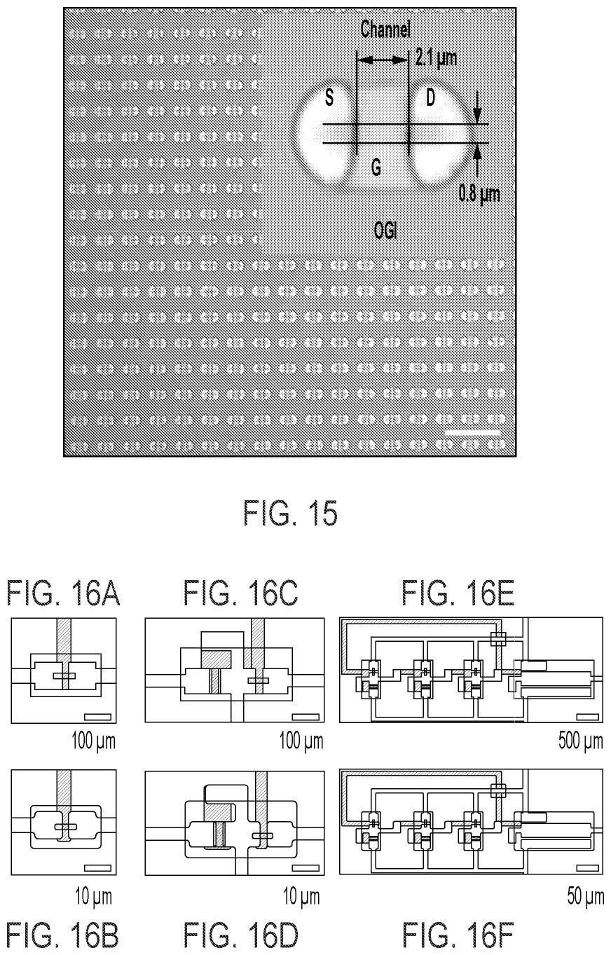

Benefiting from high effective patterning resolution (EPR), SP-2 enables down-scaled fabrication of organic devices approaching sub-micron scale. OTFT, P-channel metal oxide semiconductor (PMOS) inverters and 3-stage ring oscillators (ROs) were fabricated in two sizes via all-photolithography. FIG. 15 illustrates an optical microscopy (OM) image of a BGBC-configurated OTFT array with high device density up to 10⁶ units/cm². The photo patterned SP-2/50 has line width as small as 0.8 μm (scale bar is 20 μm). FIGS. 16A-16F illustrate OM images of down-scaled OTFTs (FIGS. 16A, 16B), PMOS inverters (FIGS. 16C, 16D), and 3-stage ring oscillators (FIGS. 16E, 16F). FIGS. 16A, 16C, and 16E are original devices, while FIGS. 16B, 16D, and 16F are shrunken devices.

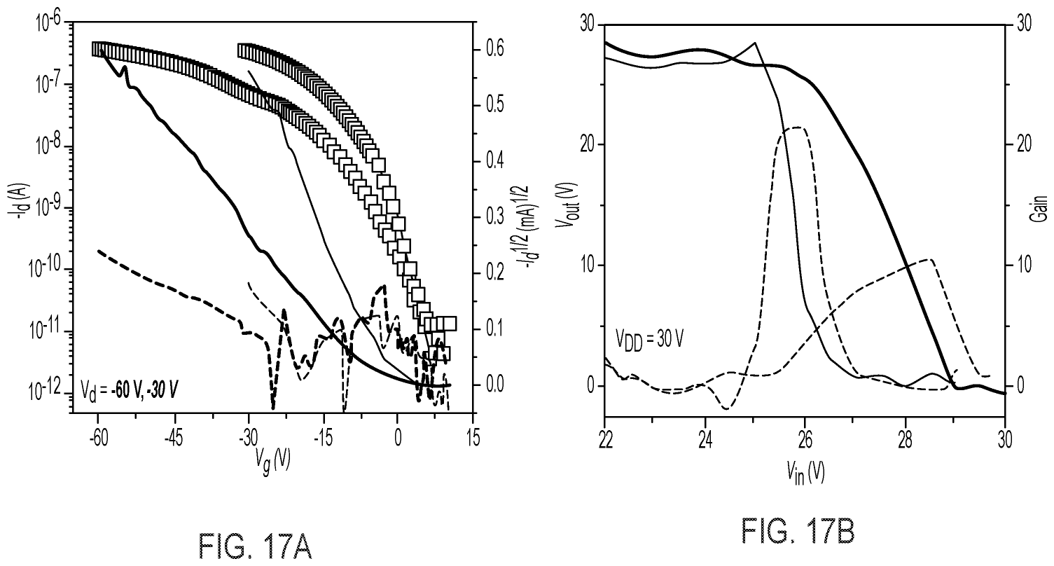

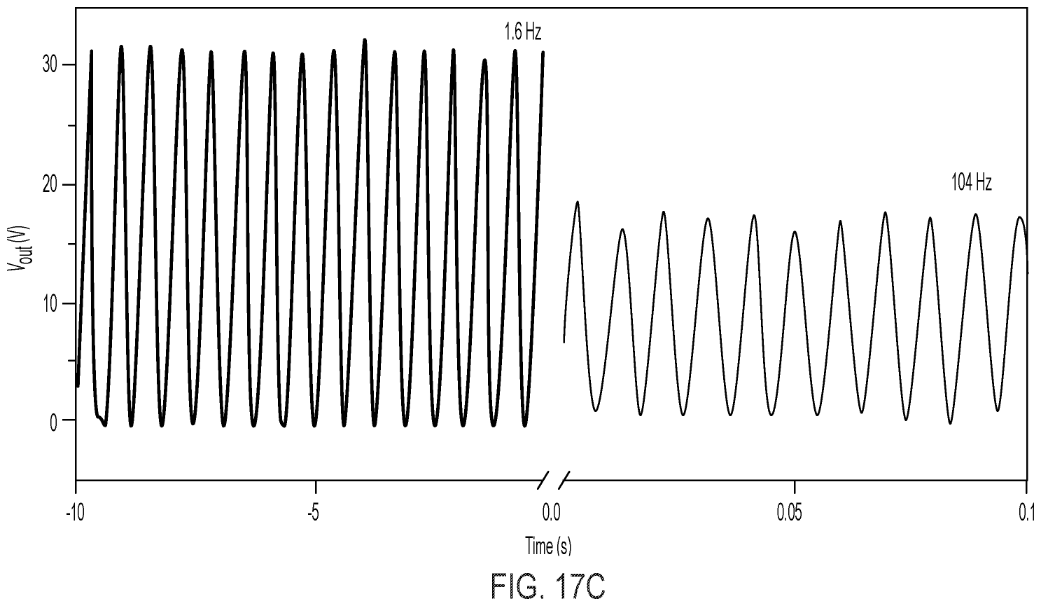

FIGS. 17A-17C illustrate electrical performance of down-scaled OTFTs (FIG. 17A), PMOS inverters (FIG. 17B), and 3-stage ring oscillators (FIG. 17C). The red line are original devices and the green line are shrunken devices. The shrunken OTFT (5×3 μm, L×W) operates at −30 V, only half the supplied voltage of the original device (50×30 μm, L×W), and maintains almost the same on-state current, achieving 50% off-power consumption. Moreover, its sub-threshold slope is (7 V dec⁻¹) is smaller than that of the original device (9 V dec⁻¹), corresponding to a faster on-off switch. In this manner, the shrunken inverters invert the electrical signal more quickly with a higher gain of 20. The original RO can only work at 60 V with a low oscillating frequency of ˜1 Hz. In contrast, the shrunken one can work at 30 V with an oscillating frequency of ˜100 Hz, which is 2 orders of magnitude higher.

### UV Patterning of Methacrylate Functionalized Semiconducting Polymer X-190401 and Alkyl Side Chain Semiconducting Polymer C255

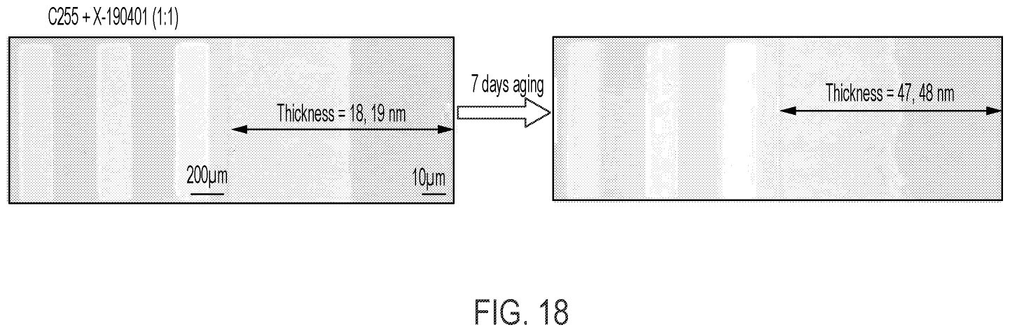

FIG. 18 illustrates an OM image of UV patterns of C255/X-190401 blend (fresh solution) (left) and an OM image of UV patterns of C255/X-190401 blend (7-day aged solution at room temperature) (right). X-190401 and C255 blends were UV-patterned under 365 nm UV mercury lamp with 400 mJ/cm². The patterns had clear edges and uniform thickness about 20 nm. The 7-day aged solution could afford UV patterns of clear edges, with increased film thickness to about 50 nm. In other words, FIG. 18, demonstrates this approach is also applicable to radical crosslinking mechanisms (X-190401), in addition to [2+2] mechanisms for cinnamate functional group.

### Example 6—General Manufacturing Procedure for OTFT Device

In examples, a bottom gate, bottom contact OTFT device can be formed as following: patterning a gold (Au) or silver (Ag) gate electrode onto a substrate, followed by spin-coating a dielectric onto the substrate and treating to obtain a gate dielectric layer. After patterning Au or Ag source and drain electrodes, an OSC layer may be formed by the materials and methods of patterning as described herein to a thickness in a range of 10 nm to 200 nm. Finally, an insulator layer was positioned. One example of the formed OTFT device is shown in FIG. 21.

In another example, a bottom gate, bottom contact OTFT device can be formed as following: patterning a gold (Au) or silver (Ag) gate electrode onto a substrate, followed by spin-coating a dielectric onto the substrate and treating to obtain a gate dielectric layer. After forming an OSC layer, Au or Ag source and drain electrodes are patterned thereon. Finally, an insulator layer was positioned. One example of the formed OTFT device is shown in FIG. 22.

Examples of organic semiconductor (OSC) polymers, crosslinkers, photoinitiators, additives, polymer blends, and device fabrication methods include, without limitation, those described in U.S. Publication No. 2022/0006016A1, UV PATTERNABLE POLYMER BLENDS FOR ORGANIC THIN-FILM TRANSISTORS, U.S. application Ser. No. 17/440,387, PHOTO-PATTERNABLE CROSS-BRED ORGANIC SEMICONDUCTOR POLYMERS FOR ORGANIC THIN-FILM TRANSISTORS, and U.S. Publication No. 2021/0367153A1, PHOTO-PATTERNABLE ORGANIC SEMICONDUCTOR (OSC) POLYMERS FOR ORGANIC THIN-FILM TRANSISTORS, each of which is assigned to Corning, Inc. and incorporated by reference herein in its entirety.

Thus, as presented herein, improved pseudo-homogeneous photo-patternable semiconducting polymer blends and use thereof for OSC layers of organic thin-film transistors are disclosed.

Semiconducting photoresist SP-2 comprising two semiconducting polymers of identical conjugated backbones enables sub-micron EPR, excellent device-to-device uniformity, and very good isotropic charge mobilities (˜0.55 cm² V⁻¹ s⁻¹) independent from channel width and processing conditions simultaneously. These promising properties may be attributed to SP-2's pseudo-homogeneous structure characterized by microphase separation down to ˜10 nm size, and isotropic short-range aggregations enhanced by the self-restrict ordering effect. Pseudo-homogeneous structure is defined meaning phase separation size of composite materials is very small, making their homogeneity close to homogeneous materials. During composite material deposition, there exists a competitive relationship between growth of different kinds of crystal domains, showing mutual inhibition, thus forming crystal domains with less order. As a result, OTFTs based on photo patterned SP-2 maintain almost unchanged charge mobilities as the channel width reduced from 50 to 1 μm. Further reducing the channel width to 0.8 μm afforded OTFT arrays with device densities up to 10⁶ units per cm², which is one order of magnitude higher than that of SP-1. In addition, SP-2 was also used to fabricate low-power consumption, high-performance organic integrated circuits, such as inverters and ring oscillators.

### Advantages

In comparison with submicron (>150 nm) phase separated SP-1, phase separation scale of SP-2 is one order of magnitude smaller. Therefore, the psudo-homogeneous blend achieves the so-far narrowest 0.8 μm channel width for photo patterned OTFTs and the highest transistor integration density up to 10⁶ units per cm².

The strong short-range isotropic aggregation and weak long-range crystallinity and orientation of SP-2 lead to very good balance between high charge mobility and isotropic device performance that is independent from channel width and centrifugal forces of spin-coating.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

As utilized herein, “optional,” “optionally,” or the like are intended to mean that the subsequently described event or circumstance can or cannot occur, and that the description includes instances where the event or circumstance occurs and instances where it does not occur. The indefinite article “a” or “an” and its corresponding definite article “the” as used herein means at least one, or one or more, unless specified otherwise.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

It will be apparent to those skilled in the art that various modifications and variations can be made without departing from the spirit or scope of the claimed subject matter. Accordingly, the claimed subject matter is not to be restricted except in light of the attached claims and their equivalents.

1. A semiconductor device, comprising:
at least one organic semiconductor (OSC) polymer and at least one photosensitizer,
wherein the at least one OSC polymer comprises a first OSC polymer and a second OSC polymer;
wherein each of the first and second OSC polymers is a diketopyrrolopyrrole-fused thiophene polymeric material, wherein the fused thiophene is beta-substituted; and
wherein each of the first and second OSC polymers comprises a repeat unit of Formula 1, Formula 2, a salt thereof, or any combination thereof: 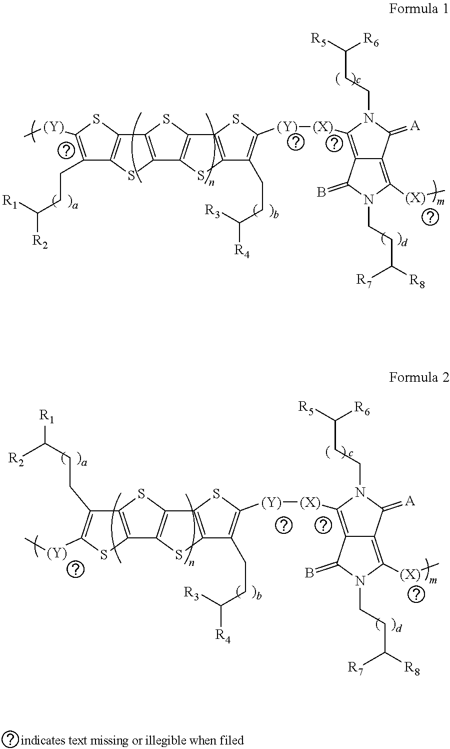
wherein:
m is an integer greater than or equal to one;
n is 0, 1, or 2;
R₁, R₂, R₃, R₄, R₅, R₆, R₇, and R₈ independently are hydrogen, substituted or unsubstituted C₄ or greater alkyl, substituted or unsubstituted C₄ or greater alkenyl, substituted or unsubstituted C₄ or greater alkynyl, or C₅ or greater cycloalkyl;
a, b, c, and d independently are integers greater than or equal to 3;
e and f independently are integers greater than or equal to zero;
X and Y independently are a covalent bond, an optionally substituted aryl group, an optionally substituted heteroaryl, an optionally substituted fused aryl or fused heteroaryl group, an alkyne or an alkene; and
A and B independently are either S or O;
with the provisos that:
(i) at least one of R₁ or R₂; one of R₃ or R₄; one of R₅ or R₆; and one of R₇ or R₈ is a substituted or unsubstituted alkyl, substituted or unsubstituted alkenyl, substituted or unsubstituted alkynyl, or cycloalkyl;
(ii) if any of R₁, R₂, R₃, or R₄ is hydrogen, then none of R₅, R₆, R₇, or R₈ are hydrogen;
(iii) if any of R₅, R₆, R₇, or R₈ is hydrogen, then none of R₁, R₂, R₃, or R₄ are hydrogen;
(iv) e and f cannot both be 0;
(v) if either e or f is 0, then c and d, independently, are integers greater than or equal to 5; and
(iv) an OSC polymer of the at least one OSC polymer has a molecular weight,
wherein the molecular weight of the OSC polymer is greater than 10,000.

2. (canceled)

3. The semiconductor device of claim 1, wherein the first OSC polymer and the second OSC polymer have identical conjugated backbones.

4. The semiconductor device of claim 1, wherein a weight ratio between the first OSC polymer and the second OSC polymer ranges from 4:1 to 1:4.

5. The semiconductor device of claim 1, comprising an isotropic charge mobility of at least 0.40 cm² V⁻¹ s⁻¹.

6. The semiconductor device of claim 1, further comprising a bottom-gate bottom-contact (BGBC)-configurated organic thin film transistor (OTFT) array.

7. (canceled)

8. The semiconductor device of claim 1, wherein at least one of R₁, R₂, R₃, and R₄ comprise acrylates, epoxides, oxetanes, alkenes, alkynes, azides, thiols, allyloxysilanes, phenols, anhydrides, amines, cyanate esters, isocyanate esters, silyl hydrides, chalones, cinnamates, coumarins, fluorosulfates, silyl ethers, or combinations thereof.

9. The semiconductor device of claim 1, wherein the at least one OSC polymer comprises a first portion and a second portion, and at least one of the first portion or the second portion comprises at least one UV-curable side chain.

10. The semiconductor device of claim 9, wherein the first portion comprises the at least one UV-curable side chain, the second portion comprises a repeat unit of Formulas 3-6 or a salt thereof: 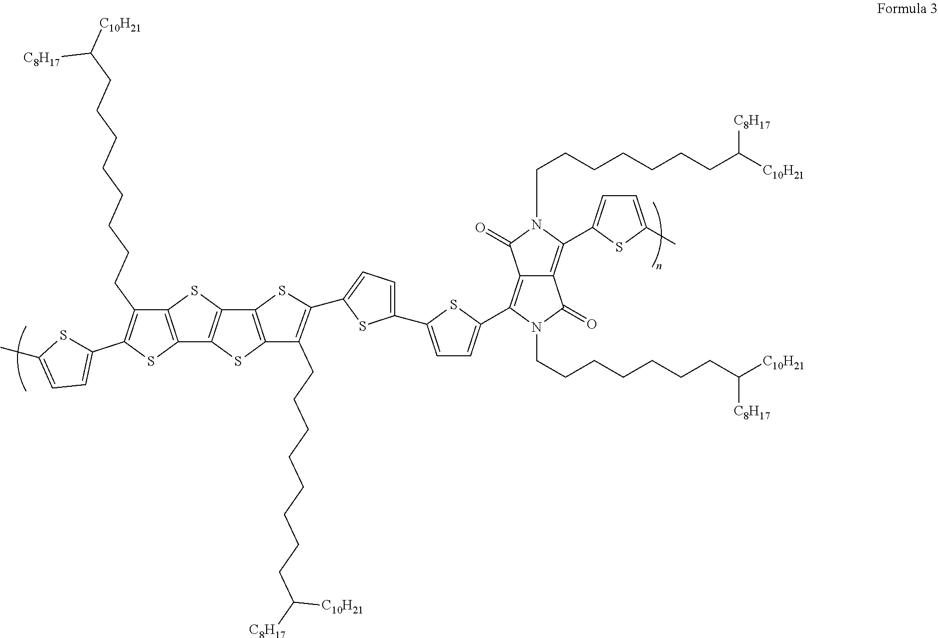 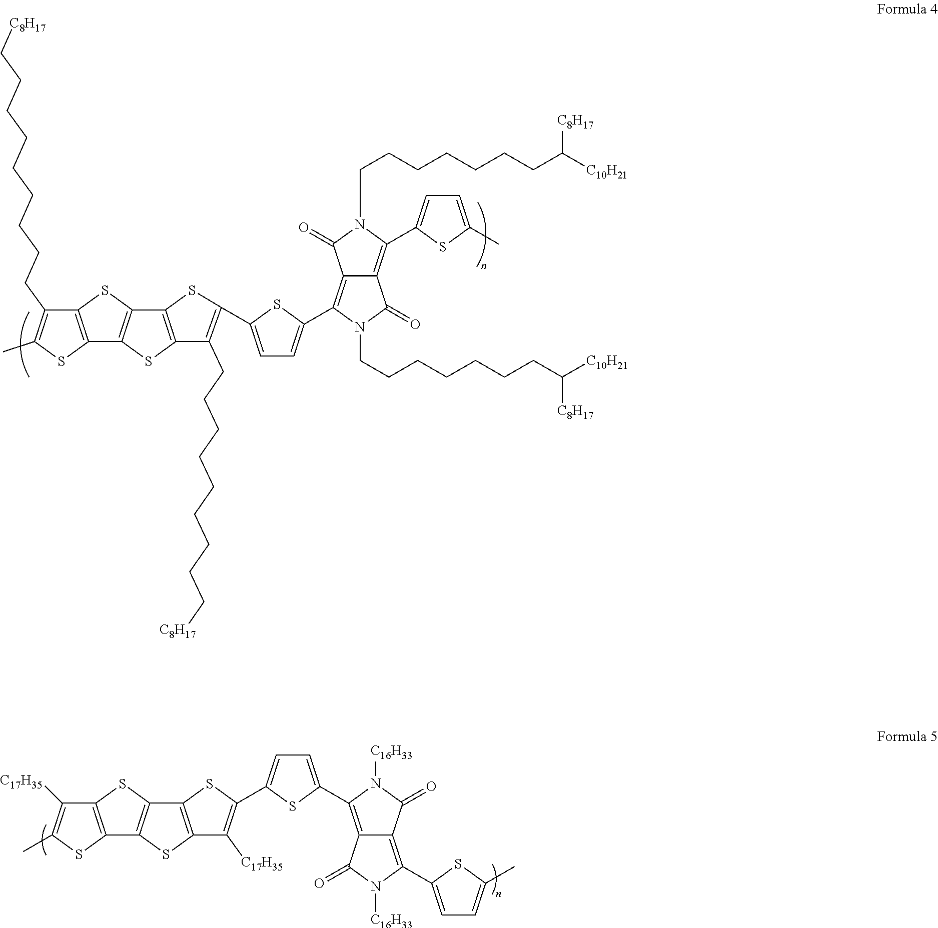 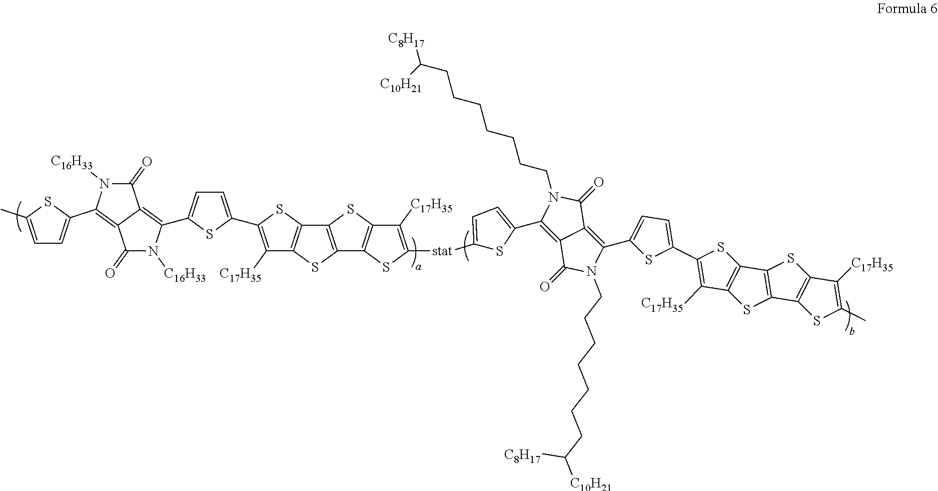
wherein:
each n in Formulas 3-5 independently is an integer greater than or equal to 1; and
a and b in Formula 6 independently are an integer greater than or equal to 1.

11. (canceled)

12. The semiconductor device of claim 9, wherein the at least one UV-curable side chain comprises at least one alkyl chain terminated by a functional group which can be UV crosslinked by a [2+2]/[4+2] mechanism.

13. The semiconductor device of claim 10, wherein the first portion comprising the at least one UV-curable side chain comprises a repeat unit of Formula 1 or Formula 1 wherein R₅ and R₇ are hydrogen and R₆ and R₈ are substituted or unsubstituted C₄ or greater alkenyl, and the second portion comprises the repeat unit of Formulas 3-6 or a salt thereof.

14. The semiconductor device of claim 1, wherein the at least one OSC polymer comprises a first portion and a second portion, wherein R₅ and R₇ are hydrogen and R₆ and R₈ are substituted or unsubstituted C₄ or greater alkenyl in the first portion and the second portion.

15. The semiconductor device of claim 1, wherein the at least one OSC polymer comprises at least one of:
a solubility of at least 0.5 mg/mL at room temperature;
hole mobility 0.1 cm²V⁻¹s⁻¹;
an on/off ratio of greater than 10⁴;
a threshold voltage in thin film transistor devices of at least −20 V.

16. The semiconductor device of claim 1, wherein the at least one OSC polymer is in a polymer blend further comprising at least one crosslinker.

17. The semiconductor of claim 16, wherein the at least one crosslinker comprises at least one of: 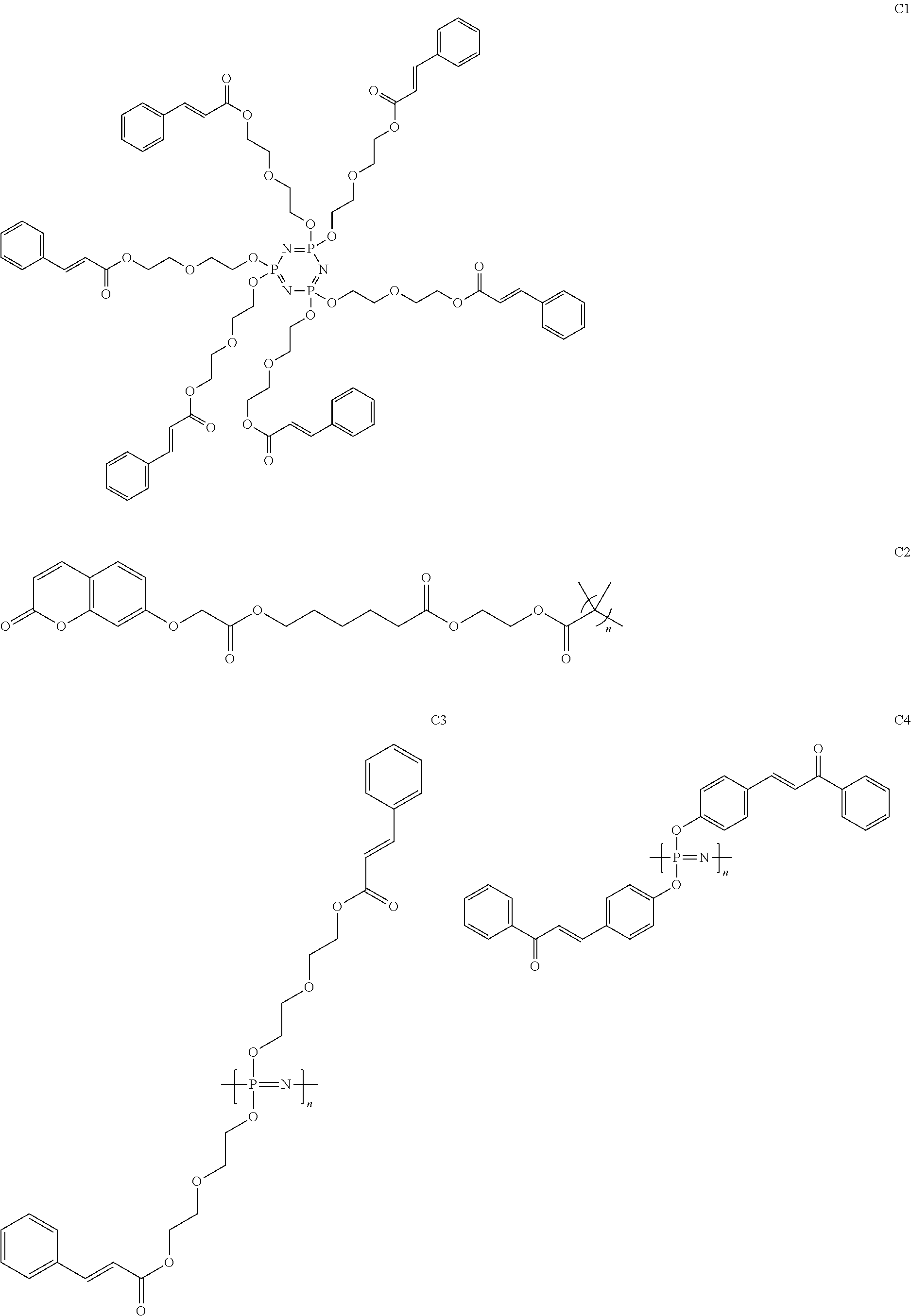 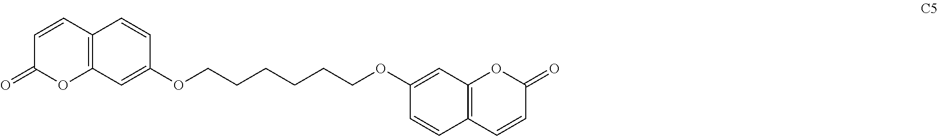 [SMILES: O=c1ccc2ccc(OCCCCCCOc3ccc4ccc(=O)oc4c3)cc2o1]
wherein each n independently is an integer greater than or equal to 1.

18. (canceled)

19. The semiconductor of claim 1, wherein the at least one photosensitizer comprises at least one of:

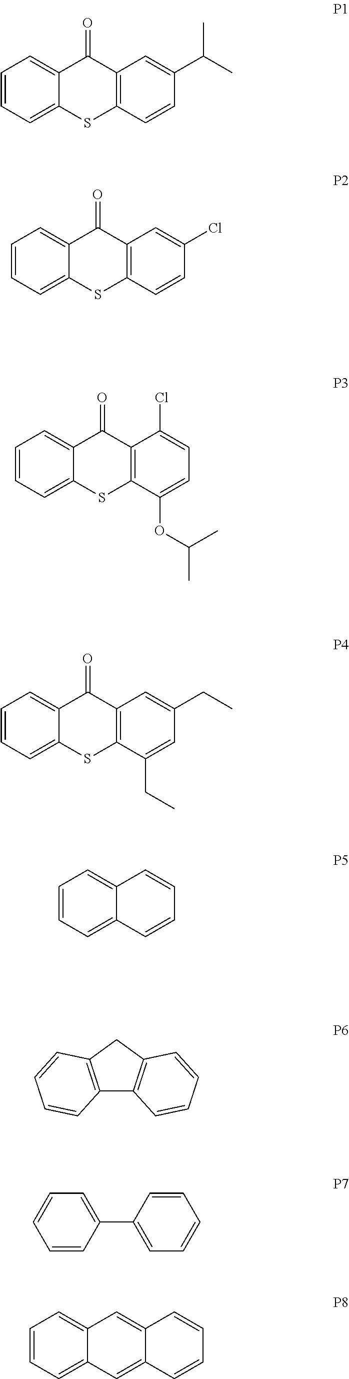

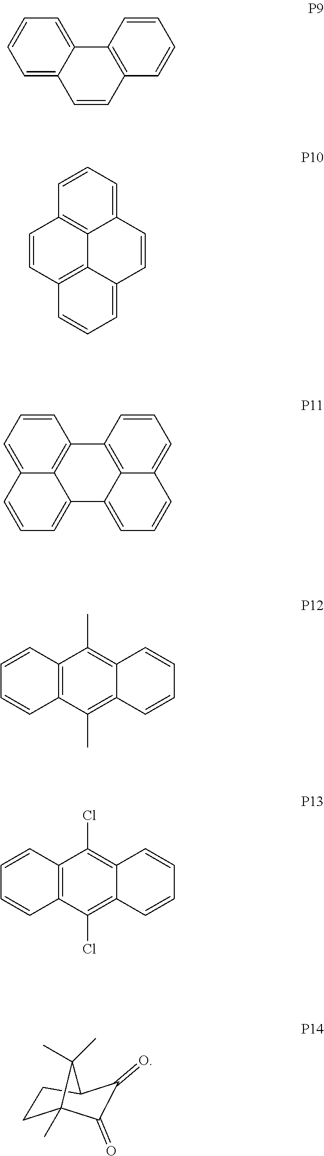

20. A method of making the semiconductor device of claim 1, the method comprising:
depositing the at least one OSC polymer on a substrate;
depositing a photoresist layer on the at least one OSC polymer;
optionally thermally annealing;
exposing the photoresist layer to UV light through a photomask to form an exposed photoresist layer;
optionally thermally baking; and
etching the exposed photoresist layer.

21. A method of making the semiconductor device of claim 1, the method comprising:
depositing the at least one OSC polymer on a substrate;
optionally thermally annealing;
exposing the photoresist layer to UV light through a photomask to form an exposed photoresist layer; and
subjecting the exposed photoresist layer to a solvent to remove un-patterned regions.
